(19) United States
(12) Patent Application Publication
(10) Pub. No.: US 2021/0146003 A1
(43) Pub. Date: May 20, 2021

(54) A NOVEL METHOD TO IMPROVE ADHESIVE STRENGTH OF REVERSIBLE POLYMERS AND HYDROGELS

(71) Applicant: UNIVERSITY OF SOUTHERN CALIFORNIA, Los Angeles, CA (US)

(72) Inventors: Mark E. THOMPSON, Los Angeles, CA (US); Mark S. HUMAYUN, Los Angeles, CA (US); John WHALEN, Los Angeles, CA (US); Niki BAYAT, Los Angeles, CA (US); Yi ZHANG, Los Angeles, CA (US); Bin LI, Los Angeles, CA (US)

(73) Assignee: UNIVERSITY OF SOUTHERN CALIFORNIA, Los Angeles, CA (US)

(21) Appl. No.: 16/623,024

(22) PCT Filed: Jun. 18, 2018
(86) PCT No.: PCT/US2018/038002

§ 371 Date: Dec. 16, 2019

Related U.S. Application Data
(60) Provisional application No. 62/520,904, filed on Jun. 16, 2017.

(51) Int. Cl. A61L 24/00; A61L 24/02; A61L 24/06; A61L 15/58; A61F 13/02

(52) U.S. Cl. CPC A61L 24/0031; A61L 24/02; A61L 24/06; C08L 33/26; A61F 13/0253; A61L 2430/16; A61L 15/58

(57) ABSTRACT

A temperature-responsive hydrogel includes water, a poly(N-alkylacrylamide) copolymer of a first monomer and a second monomer that is different than the first monomer, and an adhesion-enhancing additive. One type of adhesion-enhancing additive is selected from the group consisting of Arg-Gly-Asp-Ser amino sequence (RGDS), 3-guanidinopropionic acid (GPA), manganese(II) chloride tetrahydrate, and combinations thereof. Characteristically, the temperature-responsive hydrogel has a failure pressure that is at least 2 times greater than a failure pressure for a base temperature-responsive hydrogel having the same composition without the adhesion-enhancing additive. Another type of adhesion-enhancing additive is selected from the family of plant polyphenols, and issued as a priming layer before deployment of the temperature-responsive hydrogel. It not only improves the adhesion of temperature-responsive polymer gels to biological tissues, but also reserves the thermal reversibility of the polymers.

### CROSS-REFERENCE TO RELATED APPLICATION

This application claims the benefit of U.S. provisional application Ser. No. 62/520,904 filed Jun. 16, 2017, the disclosure of which is incorporated in its entirety by reference herein.

### STATEMENT REGARDING FEDERALLY SPONSORED RESEARCH OR DEVELOPMENT

The invention was made with Government support under Contract No. W81XWH-12-1-0314 awarded by the Army Medical Research and Material Command. The Government has certain rights to the invention.

### TECHNICAL FIELD

In at least one aspect, the present invention is related to hydrogel compositions for treating tissue injuries.

### BACKGROUND

Scleral perforation due to surgical procedure or ocular trauma occurs with a prevalence not reflected in the effectiveness or elegance of current treatment options. At least 2.5 to 3 million eye injuries occur in the United States each year, 40 to 60 thousand of which result in irreversible visual impairment. Open globe injuries account for 10% of these injuries, and despite a decline in the occurrence of superficial trauma, there has been no significant decrease in occurrence of these more serious injuries. In fact, combat-related eye injury increased up to 13% of all traumas during the US campaigns in the Middle East due to the prevalence of improvised explosive devices (IEDs). Of even greater concern is that 20-40% of battlefield ocular injuries penetrate the sclera. These injuries, while not life threatening, can have a severe impact on the life of a patient and invoke an urgent need for delivery of the best outcomes.

Not only are ocular trauma frequent, patient outcomes depending on the immediacy of treatment, especially in the case of scleral perforation. Decades of military studies and clinical observations have shown that treatments at or closest to the time of injury have the best outcomes when dealing with severe trauma to the sclera. Unfortunately, open globe injuries can require large incisions to remove foreign bodies, and may be accompanied by significant loss of scleral tissue. While several treatment methods are well established—sutures and various adhesives—traumatic loss of scleral tissue can result in irregular edge apposition that prevents watertight closure with current treatments. The sustained loss of intraocular pressure (IOP) and scleral rupture of an ineffective closure damages choroidal vasculature on the inner surface of the sclera, inviting retinal detachment and subsequent vision loss. Even successful application of those technologies invites unpleasant and unnecessary complications. As a result, there is strong demand for a straightforward technology to occlude open globe injury that can be applied by far-forward medical personnel.

Scleral penetrations and perforations are a class of open globe injuries where the sclera (the white portion) of the eye is compromised either by a single point of entry/exit (penetration) or by paired entry and exit wounds (perforations). The eye itself is a hollow globe filled with transparent fluids contained under pressure. Normal ranges for this intraocular pressure (IOP) in humans range from 10 to 20 mm Hg. Penetrating injuries lead to the release of this internal fluid and a concomitant drop in IOP.

The interior wall of the eye in the posterior segment is lined with the retina, the thin membrane-like, neurosensory tissue which transduces light images into neural signals. Stable intraocular pressure from 10 to 20 mmHg (IOP) helps to maintain the retina affixed to the interior surface of the posterior wall of the eye. This is important because the retina's photoreceptors, which transduce light photons into neural signals, are opposed with the interior surface of the eye wall and receive their metabolic support from the choroidal vasculature.

Open globe injuries to the sclera expose the posterior segment of the globe to the external environment, and compromise the internal pressure of the eye. Exposure to the external environment increases the likelihood of infection. More critically, sustained hypotony (low IOP) induced by the wall breach can lead to retinal detachment and subsequent vision loss. Although contraindicated to perform the measure, clinical reports of open globe injuries cite IOP values ranging from 0 to 4 mmHg

Several treatment methods are well established—sutures and various adhesives—the traumatic loss of scleral tissue may result in irregular edge apposition that prevents watertight closure with current treatments. Even successful application of those technologies invites unpleasant and unnecessary complications. As a result, a large body of research is devoted to rapidly deployable temporary interventions to replace a flawed standard of care.

The current standard of care for large open globe injuries is to draw the tissue margins closed with resorbable or non-resorbable sutures. This procedure is performed using a microscope and microsurgical instruments. While effective, sutures can lead to discomfort. Suture knots on the exterior surface of the eye can be abrasive and uncomfortable, leading to eye rubbing and subsequent irritation and infection, prolonging treatment. Beyond discomfort, prolonged healing times and fibrosis associated with ocular sutures have also been reported.

The shortcomings of use of sutures have inspired novel sutureless approaches to closure. Outside of the U.S., certain bio-adhesives are already approved for clinical application. Fibrin matrix sealant derived from amniotic tissue is an example. Through light-activated polymerization using Rose Bengal dye, patches of decellularized fibrin patches can be applied across the margins of lacerations on cornea. This is exciting and innovative work, however we see two concerns that may arise with commercializing this technology: 1) the use of biological tissue will likely result in protracted safety evaluation studies and increase manufacturing costs with respect to quality testing and 2) while the patch can be removed using force it is more likely to be considered irreversibly attached and thus would relegate this product to a permanent device status, also requiring more rigorous review by the FDA. A similar argument can be made against fibrin glues.

Cyanoacrylates (e.g. crazy glue) are also used outside the U.S. for ocular tissue closure and off-label in the U.S., but with mixed results. While only FDA-approved for certain tissue applications, it is known to be antibacterial and has demonstrated the ability to arrest keratolysis. However, it is non-biodegradable, sometimes difficult to dispense, irreversibly attached once applied, and requires additional tissue/substrate material in the event of traumatic injuries concomitant of significant ocular tissue loss. Cyanoacrylate also polymerizes in high modulus, rigid aggregates. The resulting solidified adhesive is granular and can feel like sand in the eye. Patient reports of “sharp edges” and ‘rocks in the eye’ demonstrate the significant oversight to patient well-being of this treatment. “Feel like ‘rocks in the eye’” testimonies from patients. Foreign body sensation is not tolerable and sharp irregular surfaces can result in erosion of surrounding tissue. Once again, we describe a treatment that can lead to discomfort and eye rubbing, which can cascade into irritation and infection.

Accordingly, a biological glue for closing wounds that prevents the deleterious effects of current technology is needed.

### SUMMARY

The present invention solves one or more problems of the prior art by providing a temperature-responsive hydrogel. The temperature-responsive hydrogel includes water, a poly(N-alkylacrylamide) copolymer of a first N-alkylacrylamide and a second monomer that is either a second N-alkylacrylamide, acrylamide, or butylacrylate, and an adhesion-enhancing additive selected from the group consisting of Arg-Gly-Asp-Ser amino sequence (RGDS), guanidine-containing compounds, manganese(II)) chloride tetrahydrate, and combinations thereof. Characteristically, the second N-alkylacrylamide when present is different than the first N-alkylacrylamide.

This invention also involves a plant polyphenol priming layer and a temperature-responsive polymer matrix. Reversible dissociation of the matrix is employed to make a reversible adhesive. The use of adhesion-enhancing additives selected from the family of plant polyphenols not only improves the adhesion of temperature-responsive polymer gels to tissues, but also reserves the thermal reversibility of the polymer gels.

The system of the present embodiment functions by leveraging the thermo-responsive behavior of a smart hydrogel. Poly(N-isopropylacrylamide) (PNIPAM) belongs to a class of intelligent aqueous polymer systems which have received great attention and popularity in a range of biomedical, drug screening, biotechnology and medical diagnostics applications. Among the family of “smart” materials, PNIPAM is the most ubiquitously studied thermo-responsive polymer. The considerable interest in PNIPAM arises because its lower critical solution temperature (LCST), and consequently its phase transition, occurs close to body temperature at 32-33° C. Below the LCST of 32° C., where water is a good solvent for polymer, PNIPAM exhibits extended conformation, high compatibility with water and poor adhesion to cells or tissue. Above the transition temperature, where polymer-polymer interactions are stronger than polymer-water interactions, PNIPAM forms a hydrophobic aggregate that readily adheres to cells and tissues.

### BRIEF DESCRIPTION OF THE DRAWINGS

FIG. 1. PNIPAM gel adhesion force test results vs. PNIPAM concentration.

FIG. 2A. Schematic showing four stages of the IOP test.

FIGS. 2B, 2C, and 2D. Scattering plots for PNIPAM compositions.

FIG. 3. Cross-sectional schematic of revised IOP test system in which hydrogel samples are tested on a dissected section of scleral tissue. Dissected sclera (a) is mounted into a modified 60 mL syringe (b) with a custom port (c) for filling with saline and insertion of the pressure sensor. Pressure is controlled using a digitally controlled infusion system connected to the syringe plunger (d).

FIG. 4. Maximum IOP maintained by different hydrogel+additive mixtures in the artificial pig eye model.

FIGS. 5A, 5B, 5C, 5D, 5E, and 5F. Intensity distribution graph of DLS spectra for aqueous N20BA1 solution at different temperatures.

FIGS. 6A, 6B, 6C, and 6D. Intensity distribution graph of DLS spectra for aqueous N20BA1 solution as a function of temperature.

FIGS. 7A and 7B. Radius variation as a function of temperature for 5% N20BA1

FIGS. 8A, 8B, and 8C. The hydrodynamic radius of aqueous N20BA1 copolymer solution with various concentrations of RGDS, as a function of temperature.

FIGS. 9A and 9B. Particle size distribution of the aggregates with various RGDS concentration.

FIGS. 10A and 10B. Variation of hydrodynamic radii of the aggregates as a function of temperature for N20BA1 (5% w/v) with different concentrations of RGDS.

FIGS. 11A, 11B, and 11C. Variation of hydrodynamic radii of aggregates as a function of temperature for N20BA1 with different concentration of GPA in solution state.

FIGS. 12A and 12B. DLS spectra of the intensity distribution graph of copolymer solution with various concentrations of GPA.

FIGS. 13A and 13B. Temperature-dependent variation of the hydrodynamic radii of N20BA1 particles prepared with different amounts of GPA.

FIGS. 14A and 14B. Variation of hydrodynamic radii of the aggregates as a function of temperature for N20BA1 with different concentrations of RGDS and GPA.

FIGS. 14C, 14D, 14E, and 14F. DLS data consistency.

FIGS. 15A, 15B, 15C, and 15D. Fluorescence intensity of ANS in pNIPAM and N20BA1 solutions at different temperature.

FIGS. 16A and 16B. Scattering spectra of the aqueous copolymer solution with various concentration of N20BA1 as a function of temperature.

FIG. 17. Variation of scattering intensity of 5% N20BA1 with different slits width.

FIGS. 18A, 18B, and 18C. Variation of scattering intensity of N20Ba1 in water at different temperatures.

FIGS. 19A, 19B, 19C, and 19D. Changes in scattering intensity of N20BA1 with increasing RGDS and GPA concentration as a function of temperature.

FIGS. 20A, 20B, and 20C. Change in scattering intensity of N20BA1 with increasing RGDS and GPA concentration at different temperature in N20BA1-additive solutions.

FIG. 21. Viscosity of various aqueous copolymer solutions as a function of temperature.

FIG. 22. Viscosity of aqueous N20BA1 (5% w/v) copolymer solution with various heating rates, as a function of temperature.

FIG. 23. The variation in the viscosity of N20BA1-RGDS solutions as a function of temperature.

FIG. 24. Temperature dependent viscosity of 5% N20BA1 (w/v) aqueous solution (black color line) and in the presence of 0.58% GPA (red color line), 1.16% GPA (blue color line) and 2.91% GPA (pink color line) as a function of temperature.

FIG. 25. RGDS and GPA effect on aqueous additive-free 5% N20BA1 (w/v) solution at different temperatures.

FIGS. 26A and 26B. Plots of loss modulus versus temperature for various PNIPAM compositions.

FIGS. 27A and 27B. Plots of storage modulus versus temperature for various PNIPAM compositions.

FIG. 28. The family of polyphenol compounds.

FIGS. 29A, 29B, 29C, and 29D. Tunability of cohesion strength of the polymer matrix by copolymerization with butyl acrylate and changing the polymer concentration.

FIGS. 30A, 30B, and 30C. Comparison of the adhesion strength of P(NIPAM100-BA5) matrix to porcine sclera using different deployment methods.

FIGS. 31A and 31B. Change of adhesion strength of P(NIPAM100-BA5) matrix to porcine sclera by varying the TA concentration and incubation time.

FIGS. 32A and 32B. Change of adhesion strength of polymer matrix to porcine sclera by varying the P(NIPAM100-BA5) concentration and incubation time.

FIG. 33. Change of adhesion strength of P(NIPAM100-BA10) matrix to porcine skin by varying the Fe(III)/TA molar ratio.

FIG. 34. The adhesive strength of P(NIPAM100-BA5) matrix to porcine sclera using TA priming layer tested at different temperatures.

FIG. 35. Validation of adhesion enhancement of P(NIPAM100-BA5) matrix to different substrates using TA priming layer.

FIG. 36. Validation of adhesion enhancement of gelatin gels to different substrates using TA priming layer.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: all R groups (e.g. R and Rᵢ where i is an integer) include alkyl, lower alkyl, C₁₋₆ alkyl, C₆₋₁₀ aryl, or C₆₋₁₀ heteroaryl; single letters (e.g., “m” “n” or “o”) are 1, 2, 3, 4, or 5; ranges of integers specifically include individual the endpoints and all intervening integers (e.g., 1-5 specifically includes each of 1, 2, 3, 4, and 5); percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

Throughout this application, where publications are referenced, the disclosures of these publications in their entireties are hereby incorporated by reference into this application to more fully describe the state of the art to which this invention pertains.

The term “alkyl”, as used herein, unless otherwise indicated, includes C₁₋₁₂ saturated monovalent hydrocarbon radicals having straight or branched moieties, including, but not limited to, methyl, ethyl, n-propyl, iso-propyl, n-butyl, sec-butyl, tert-butyl, and the like.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

The term “(meth)acrylic” used herein includes both acrylic and methacrylic and the term “(meth)acrylate” includes both acrylate and methacrylate. Likewise, the term “(meth)acrylamide” refers to both acrylamide and methacrylamide. “Alkyl” includes straight chain, branched and cyclic alkyl groups.

The term “alkyl” refers to C₁₋₂₀ inclusive, linear (i.e., “straight-chain”), branched, saturated or at least partially and in some cases fully unsaturated (i.e., alkenyl and alkynyl) hydrocarbon chains, including for example, methyl, ethyl, n-propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. Preferably the alkyl groups used here are C₁₋₆ alkyl.

The term “alkoxy” means a straight or branched-chain alkoxy group. Typically, alkoxy has 1 to 6 carbon atoms (i.e., C₁₋₆ alkoxy) containing a Examples of alkoxy are methoxy, ethoxy, propoxy, isopropoxy, butoxy, t-butoxy and the like.

The term alkylalkoxy means a combination of an alkyl or substituted alkyl group and an alkoxy or substituted alkoxy group. Typically, alkylalkoxy has 2 to 10 carbon atoms (i.e., C₂₋₁₀ alkoxy)

The term “aryl” means a C_{6_18} aromatic carbocyclic ring or ring system, which is unsubstituted or substituted by one or more (e.g., 1-3) substituents. Examples of substituents are C₁₋₆ alkyl, hydroxy, C₁₋₆ alkoxy, and halogen. Examples of aromatic carbocyclic rings are phenyl and naphthyl.

The term “heteroaryl” means a C₄₋₁₈ aromatic a heterocyclic ring or ring system, which is unsubstituted or substituted by one or more (e.g., 1-3) substituents. Examples of substituents are C₁₋₆ alkyl, hydroxy, C₁₋₆ alkoxy, and halogen. Examples of aromatic carbocyclic rings are phenyl and naphthyl. Examples of aromatic heterocyclic rings are pyridino, pyrrolo, thienyl, pyrazalo, imidazalo, thiazalo, oxazalo, triazalo, teatrazalo, oxadiazalo, thiadiazolo, benzofuryl, benzothienyl, benzinidazalo, benzotriazalo, quinololyl, isoquinolyl, and indolyl.

Abbreviations:

“AM” means acrylamide.

“BA” means butyl acrylate.

“EGC” means epigallocatechin.

“EGCG” epigallocatechin gallate epicatechin gallate.

“GA” means gallic acid.

“IOP” means intraocular pressure.

“LCST” means lower critical solution temperature.

“PNIPAM” means poly(N-isopropylacrylamide).

“NEAM” means N-ethylacrylamide.

“NMAM” N-methylacrylamide

“NNBAM” means N-n-butylacrylamide.

“NTBAM” means N-t-butylacrylamide.

“TA” means tannic acid.

“UCST” means upper critical solution temperature.

In an embodiment, a temperature-responsive hydrogel includes water; a poly(N-alkyacrylamide) copolymer, and an adhesion-enhancing additive. In a variation, the poly(N-alkyacrylamide) copolymer is a copolymer of a first monomer having formula 1 and a second monomer that is different than the first monomer. In a refinement, the second monomer has formula 2:

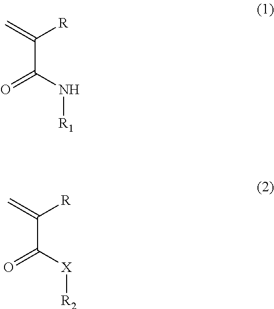

wherein R is H or C₁₋₆ alkyl (e.g., methyl); R₁ is —(CH₂)ₙ—R₃, C₁₋₆ alkyl, C₆₋₁₈ aryl, or C₄₋₁₈ heteroaryl; R₂ is H, C₁₋₆ alkyl, C₆₋₁₈ aryl, or C₄₋₁₈ heteroaryl; R₃ is H, hydroxyl, F, Cl, Br, NH₂, or N(R₄)₂; R₄ is H or C₁₋₆ alkyl; n is an integer from 0 to 6 (i.e., 0, 1, 2, 3, 4, 5 or 6) and X is O or NH. The adhesion-enhancing additive selected from the group consisting of Arg-Gly-Asp-Ser amino sequence (RGDS), guanidine-containing compounds, manganese(II) chloride tetrahydrate, and combinations thereof. In a refinement, R₁ and R₂ are each independently methyl, ethyl, n-propyl, iso-propyl, n-butyl, sec-butyl, or tert-butyl. In a particularly useful refinement, R₁ is iso-propyl. Typically, the weight ratio of the first monomer to the second monomer is from about 99:1 to about 50:50.

A particularly useful example for the first monomer is N-isopropylacrylamide. Examples for the second monomer include, but are not limited to, acrylamide, N-ethylacrylamide, N-methylacrylamide, N-n-butylacrylamide and N-t-butylacrylamide. Advantageously, the temperature-responsive hydrogel of the present embodiment has a failure pressure that is at least 2 times greater than a failure pressure for a base temperature-responsive hydrogel having the same composition without the adhesion-enhancing additive. In a refinement, the temperature-responsive hydrogel of the present embodiment has a failure pressure that is at least 3, 4, or 5 times greater than a failure pressure for a base temperature-responsive hydrogel having the same composition without the adhesion-enhancing additive. In another refinement, the temperature-responsive hydrogel of the present embodiment has a failure pressure that is 2 to 6 times greater than a failure pressure for a base temperature-responsive hydrogel having the same composition without the adhesion-enhancing additive. In this context, failure pressure is the pressure (e.g., intraocular pressure) at which a patch formed from the temperature-responsive hydrogel gives way. At this pressure, ocular fluid leaks from the eye either through the patch or around it. It is observed that in most cases where increasing pressure leads to a leak, lowering it below the critical pressure reseals it. When the pressure is raised enough that the patch fails it is ruptured and will fail at all pressures after that.

Examples of guanidine-containing compounds include, but is not limited to, aganodine, agmatidine, agmatine, ambazone, amiloride, apraclonidine, aptiganel, argatroban, arginine, argininosuccinic acid, asymmetric dimethylarginine, benexate, benzamil, bethanidine, BIT225, blasticidin s, brostallicin, camostat, cariporide, chlorophenylbiguanide, cimetidine, ciraparantag, creatine, creatine ethyl ester, creatine methyl ester, creatinine, creatinolfosfate, 2-cyanoguanidine, cycloguanil, debrisoquine, dihydrostreptomycin, ditolylguanidine, E-64, ebrotidine, epinastine, eptifibatide, famotidine, glycocyamine, guanabenz, guanadrel, guanazodine, guanethidine, guanfacine, guanidine, guanidine nitrate, guanidinium chloride, guanidinium thiocyanate, 5′-guanidinonaltrindole, 6′-guanidinonaltrindole, guanidinopropionic acid, guanochlor, guanoxabenz, guanoxan, gusperimus, impromidine, kopexil, laninamivir, leonurine, lombricine, lugduname, metformin, methylarginine, mitoguazone, octopine, OUP-16, pentosidine, peramivir, phosphocreatine, picloxydine, pimagedine, polyhexamethylene guanidine, n-propyl-l-arginine, rimeporide, robenidine, saxitoxin, siguazodan, streptomycin, sucrononic acid, sulfaguanidine, synthalin, TAN-1057 A, TAN-1057 C, tegaserod, terbogrel, 1,1,3,3-tetramethylguanidine, tetrodotoxin, tomopenem, triazabicyclodecene, UR-AK49, vargulin, VUF-8430, and zanamivir. A particularly useful guanidine-containing compound is 3-guanidinopropionic acid (GPA).

The temperature-responsive hydrogel also provides a number of additional advantages over base temperature-responsive hydrogels having the same composition without the adhesion-enhancing additive. For example, the temperature-responsive hydrogel have a reduced viscosity over the temperature range 2-26 C. In particular, the viscosity can be reduced by 30 percent or more over this range. In addition, the sized of aggregates formed in the temperature range 12-18° C. is higher in general for the temperature-responsive hydrogel as compared to base temperature-responsive hydrogels having the same composition without the adhesion-enhancing additive.

The present embodiment is not limited by any particular amounts for its components. In one variation, the poly(N-alkyacrylamide) copolymer is present in an amount of about 0.5 weight percent to about 50 weight percent of the total weight of the temperature-responsive hydrogel. In another variation, the poly(N-alkyacrylamide) copolymer is present in an amount of about 10 weight percent to about 60 weight percent of the total weight of the temperature-responsive hydrogel. Typically, the adhesion-enhancing additive is present in an amount of about 0.01 weight percent to about 25 weight percent of the total weight of the temperature-responsive hydrogel. In each of these temperature-responsive hydrogel compositions, the balance is water.

Although the present embodiment is not significantly limited by the molecular weight of the a poly(N-alkyacrylamide) copolymer, typically, the poly(N-alkyacrylamide) copolymer, has a number average molecular weight of about 5,000 to about 5,000,000 Daltons. In a refinement, the poly(N-alkyacrylamide) copolymer has a number average molecular weight of about 10,000 to about 3,000,000 Daltons. In still another refinement, the poly(N-alkyacrylamide) copolymer has a number average molecular weight of about 20,000 to about 2,000,000 Daltons.

In some variations, the poly(N-isopropylacrylamide) copolymer is a block copolymer. In other variation, the poly(N-isopropylacrylamide) copolymer is a statistical or random copolymer.

The present embodiment represents an improvement to the hydrogel of U.S. Pat. Pub. No. 2016/0220725; the entire disclosure of which is hereby incorporated by reference and attached as Exhibit A. Therefore, the temperature-responsive hydrogel may also contain one or more excipients, stabilizers, additives or the like. The instant hydrogels may also comprise a bioactive agent, a diagnostic agent, a cosmetic agent, colorant (to enhance visualization), or any other agent suitable for delivery to the eye. For example, in one or more embodiments, the hydrogel may comprise a therapeutically effective amount of a bioactive agent. Representative active agents include but are not limited to, for example, antibiotics, anti-inflammatory agents, chemotherapeutic agents, steroids, and immunosuppressants. Moreover, the temperature-responsive hydrogel can be applied through an adhesive patch on a polymeric substrate (e.g., parylene, poly-lactic acid, polyimide, and polydimethylsiloxane). In some refinements, the temperature-responsive hydrogel has an adhesive strength of in a range between 10 mN to 10,000 mN when measured using an in vitro uniaxial adhesion test to scleral tissue at 370° C.

In another variation, the poly(N-alkyacrylamide) copolymer is a copolymer of a first monomer having formula 1, a second monomer (e.g., formula 2) that is different than the first monomer and one or more additional monomers having formula 3 that is different than the first monomer and second monomer:

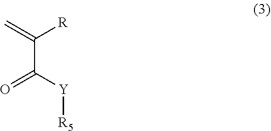

where Y is O or NHR₆, R is H or C₁₋₆ alkyl; R₅ is —(CH₂)ₘ—R₇; R₆ is H or C₁₋₆ alkyl; R₇ is halo, hydroxyl, C₆₋₁₂ aryl, C₄₋₁₈ heteroaryl, amino, phosphorylcholinyl having formula 4, pyridinyl and the like; m is an integer from 0 to 18 or 1 to 18:

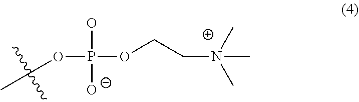

In particular, R₅ is —CH₂—R₇ or —CH₂CH₂—R₇. An example of a R₅ is dodecyl pyridinyl (—C₁₇H₂₉N) having formula 5:

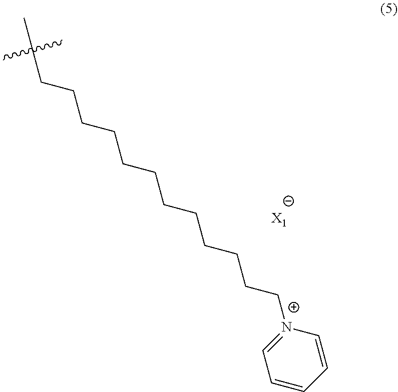

where X₁⁻ is a counterion such as halide (e.g., F—, Cl—, Br—). Advantageously, these additional monomers having be used to modify physical and adhesive properties of the temperature-responsive hydrogel set forth above

In another embodiment, a method for reversibly sealing tissue damage is provided. The method includes a step of applying the temperature-responsive hydrogel set forth above to a tear or perforation in a tissue of a subject in an amount effective to seal the tear or perforation. The tissue can be ocular tissue (e.g., cornea, sclera), skin, mucosal tissue, etc. Characteristically, when the temperature-responsive hydrogel is exposed to a temperature above its critical solution temperature, the hydrogel becomes adhesive, and when exposed to a temperature below its critical solution temperature, the hydrogel becomes less adhesive. Therefore, the hydrogel is usually maintained at a temperature below its critical solution temperature prior to application because of the lower viscosity at such temperatures. Moreover, the temperature of the ocular tissue is usually above the critical solution temperature of the hydrogel. When applied, the temperature-responsive hydrogel advantageously adheres to the tissue of the edges of the tear. In a refinement, in order to ensure proper sealing of the ocular tear, the temperature-responsive hydrogel can be applied to an inner surface of the eye wall a slight excess of an amount of hydrogel effective to fill a void created by the ocular tear. In this latter refinement, the ocular pressure is effective to press the excess hydrogel against the inner surface of the eye to thereby create an internal ocular seal.

In another embodiment, a two-step method for reversibly sealing tissue damage using a primer layer is provided. The two-step method includes a step of applying a priming layer to a tear or perforation in in a tissue of a subject. The tissue can be ocular tissue (e.g., cornea, sclera), skin, mucosal tissue, etc. The priming layer is formed from a polyphenol solution that includes the polyphenol. A polymer solution is then applied to the tear pre-coated with the prior layer in an amount effective to form a sealing layer that seals the tear. The polymer solution includes a temperature-responsive polymer (e.g., a hydrogel as set forth above). The polymer solution can be any composition having the requisite properties. In one variation, the polymer solution is the temperature-responsive hydrogel is that set forth above with or without the additives. In a variation, the priming layer includes polyphenols and in particular, plant polyphenols. In this embodiment, temperature is used as a direct or indirect trigger, reversible dissociation of the matrix is employed as a new concept of reversible adhesive design.

Specifically, the two-step method includes a polyphenol solution that is applied on the target substrate or tissue, forming a priming layer. After washing away the unbonded polyphenol with neutral or weakly alkaline water, a polymer solution is applied the primed substrate or tissue. As the polymer transits to a gel or elastomer state over a critical temperature, a strong bond is formed at the interface. On demand release can be triggered simply by changing the temperature, either cooling or heating depending on the polymer type, the polymer matrix will lose all cohesive strength by marked softening or dissolution. The substrate to adhere can be biological tissues, including but not limited to skin, cornea, sclera and mucosal surface, hydrogels and any other soft substrates.

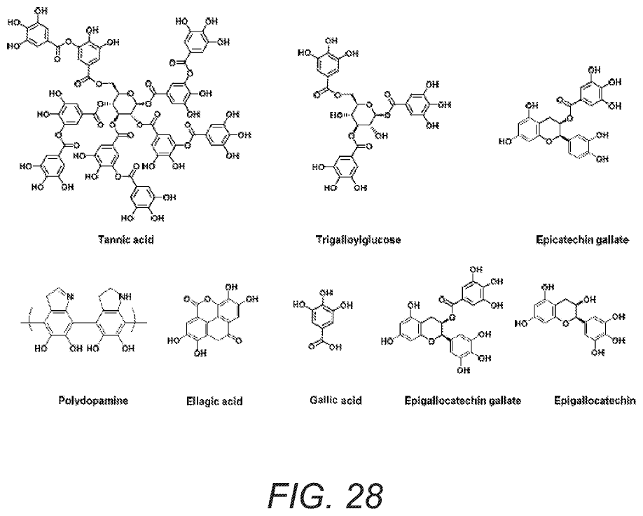

In a refinement, the priming layer includes polyphenol compounds or residues thereof. The polyphenol compounds used in this invention is derived from nature, and consist of a large family of compounds with dihydroxyphenyl (catechol) and trihydroxyphenyl (gallic acid, GA) residues. These compounds are commercial available or can be easily synthesized from commercial materials, and usually biocompatible, biodegradable, and widely used as food additives. Members of the family include tannic acid (TA), polydopamine, epigallocatechin gallate (EGCG), epicatechin gallate (ECG), epigallocatechin (EGC), ellagic acid and trigalloylglucose, to name a few (FIG. 28). They contribute to strong adhesion through multiple intermolecular interactions with polymers, including hydrogen bonding, electrostatic interaction, hydrophobic interaction and covalent bonding, and serves as a ‘molecular glue’.

The polymer matrix used for the invention must meet two requirements. First, it's a thermoresponsive polymer in aqueous solution, it is in a liquid state and applicable at one temperature, and transitions to a solid state at the target temperature. Second, the solid state, in the form of gels or elastomers, above the critical temperature must possess a good cohesion strength. The polymers include two series of polymers showing opposite phase behaviors. One is the polymers showing a coil-to-globule transition above its lower critical solution temperature, for example, poly(N-isopropylacrylamide) copolymers. These polymers will have little or no cohesive strength below their LCST. Another are the polymers that gels upon cooling, including many natural polymers such as gelatin, agarose, gellan gum, xyloglucan and k-carrageenan and synthetic polymer with UCST-type behaviors such as poly(N-acryloylglycinamide) and copolymers, which will lose cohesive strength on heating.

Advantageously, the adhesive performance of the coating formed from the of the two-step method is tunable. The transition temperature and adhesion/cohesion strength of synthetic polymer matrix can be varied by changing the polymer concentration, molecular weight and dispersity, and copolymerization with hydrophobic/hydrophilic comonomers. The transition temperature and adhesion/cohesion strength of natural polymer matrix can be varied by changing specifications of the polymers, further improvement can be achieved by physical mixing and chemical modification.

Extra control of the adhesion strength includes the deployment method, the incubation time, the polyphenol concentration, and the coordination of polyphenol with multivalent metal, mainly transition metal ions such as Fe(III).

The release trigger can be switched to light by incorporating a photothermal agent. It includes inorganic materials (i.e., photothermal elements) such as gold nanoparticles, copper sulfide crystals, carbon nanomaterials, and black phosphorus. After entrapped within the thermoresponsive polymer matrix, photothermal agents will emit heat with light illumination, realizing remote/spatial control of adhesion.

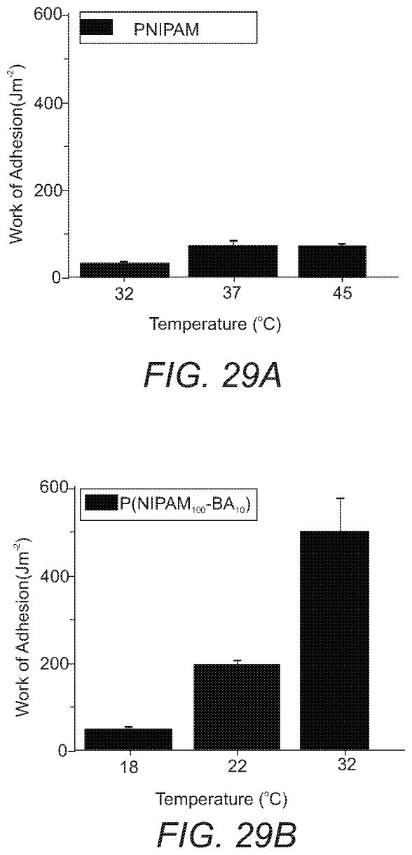

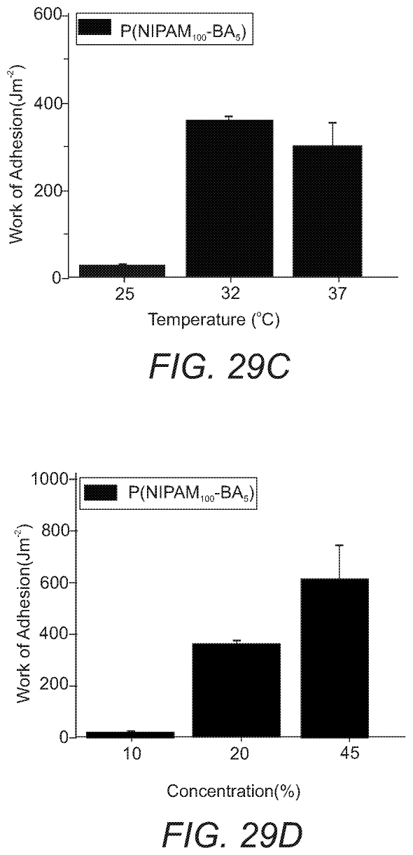

The cohesion strength of PNIPAM copolymer matrix used in the two-step method is determined by temperature, which is the basis of the reversible adhesive design using cohesion failure. The enhancement of cohesion strength above critical temperature can be realized by copolymerization of NIPAM with a hydrophobic monomer, butyl acrylate (BA). Changing the feeding ratio of BA can shift the transition temperature, to accommodate applications at different temperature, and the polymer concentration can be varied to get different cohesion strength at the same temperature. FIG. 29 illustrates the tunability of cohesion strength of the polymer matrix by copolymerization with butyl acrylate and changing the polymer concentration.

The following examples illustrate the various embodiments of the present invention. Those skilled in the art will recognize many variations that are within the spirit of the present invention and scope of the claims.

This section reports the design, fabrication and preliminary in vitro validation of a novel system for temporary intervention of open globe injuries to the sclera for potentially improving visual outcomes for ocular trauma patients. The concept is based on a thermo-responsive hydrogel which can be spread over the penetrating injury as a fluid and as it heats with the patient's body temperature the hydrogel increases in viscosity leading to formation of a solid patch to occlude the penetrating injury. This program builds on existing work in hydrogels for biomedical applications by synthesizing and characterizing a novel series of thermo-responsive co-polymers to precisely tailor the temperature response of the smart material to optimize performance for occluding injuries. Adhesion data performed on preliminary samples under uniaxial testing showed that the strength of attachment to scleral tissue (porcine) in vitro was significantly lower than cyanoacrylate (a commonly used and FDA approved tissue adhesive for other clinical applications).

Research was refocused on adjusting chemistry to improve adhesive performance. Different solution chemistries were prepared and compared in a uniaxial tension test to track any performance improvements. Chemistries showing improved adhesion would move on to in vitro IOP testing.

Hydrogel Synthesis

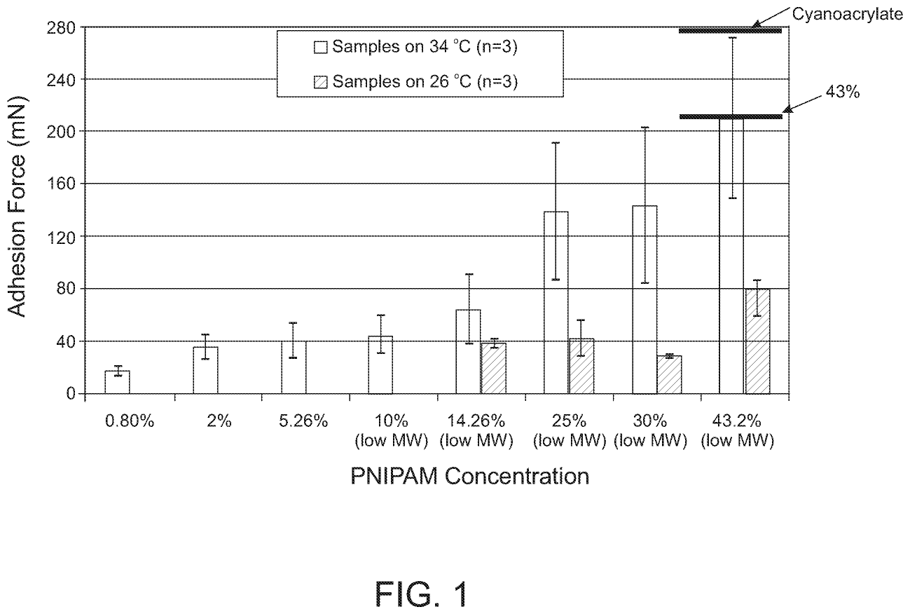

It is observed that increasing concentration of PNIPAM in the solution has a significant positive impact on adhesion. Different PNIPAM hydrogels were prepared by varying aqueous hydration from 0.8% to 43.2% hydration. When tested in uniaxial tension, it was found that the adhesion increased with increasing hydration. However, when compared to cyanoacrylate tested in the same linear pull test, it was found that 43% aqueous PNIPAM exhibited only 80% of the adhesion strength of cyanoacrylate to scleral tissue (see, FIG. 1)

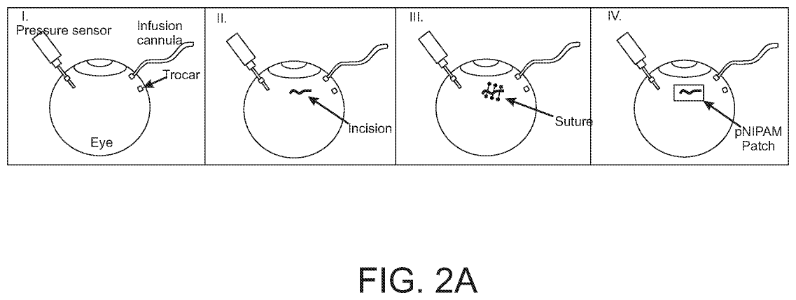

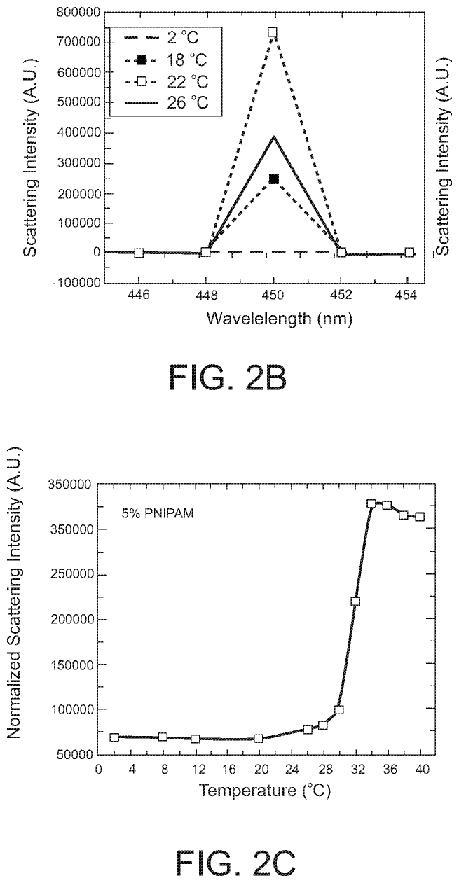

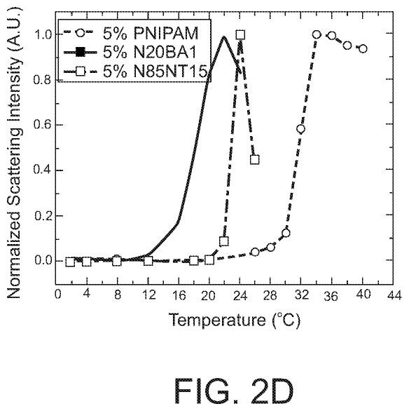

The 43% PNIPAM, was tested in the IOP test protocol to measure ability to arrest leakage under ocular pressure conditions (see, FIG. 2). Infusion rates for pre-incision, post-incision creation, post-suturing and post hydrogel placement are shown in Table 1. While leak rates were lower than any other sample tested to date, these results still show leakage. IOP Test results for 43% PNIPAM did not meet success criteria (complete arrest of saline infusion at 16 mm Hg pressure).

TABLE 1
Saline Infusion Rates in Porcine Eye Model of Scleral Penetration

| | Infusion Flow Rate (cc/min) | | | |
|---|---|---|---|---|
| Adhesive Material Tested | I. | II. | III. | IV. |
| Cyanoacrylate Glue | 0 | 18 | 0 | 0 |
| 43.2% PNIPAM at 16.5 mm Hg | 0 | 19 | 0 | 12 |
| 10% (85% PNIPAM: 15% n-tert ButylA) | 0 | 10 | 0 | 5 |
| 10 mm Hg | | | | |
| 10% (85% PNIPAM: 15% n-tert ButylA) | 0 | 13 | 0 | 7.5 |
| 20 mm Hg | | | | |

The phase transition property of PNIPAM can be tuned by making numerous copolymers of PNIPAM where the choice of co-monomer, its fraction, and its hydrophilicity alter the LCST. For instance, Priest et al. reported phase transition behavior for a series of copolymers of NIPAM with other hydrophilic and hydrophobic N-alkylacrylamides such as acrylamide (AM), N-ethylacrylamide (NEAM), N-methylacrylamide (NMAM), N-n-butylacrylamide (NNBAM) and N-t-butylacrylamide (NTBAM). They observed that increasing the amount of a hydrophilic co-monomer (e.g. AM, NEAM and NMAM) in the copolymer increases the LCST. They also reported that copolymers of NIPAM with relatively hydrophobic co-monomers (e.g. NTBAM) exhibited decreases in LCST as a linear function of the co-monomer input ratio.

Co-polymers of NIPAM and NNBAM showed similar behavior up to 40% NNBAM, but the LCST sharply decreased from 17° C. to below zero with higher percentages. The n-butyl group was hypothesized to associate and precipitate more readily than less flexible t-butyl groups. Natalia et al. have confirmed that a copolymer of NIPAM and butylacrylate has lower LCST compared to PNIPAM, which is attributed to the presence of relatively hydrophobic butylacrylate (BA) segments in the copolymer.

Butylacrylate seems to induce an increase of hydrophobic interactions between hydrophobic isopropyl groups in NIPAM and butyl groups in butylacrylate, thus causing copolymer collapse at lower temperature. The hydrophobic NTBAM and BA not only decrease the LCST of PNIPAM, but also improve the polymer's mechanical properties and cell adhesion. Below transition temperature, the hydrophilic characteristic of copolymer induces bonding interactions with water molecules rather than with cell protein, therefore cell detachment from substrate occurs. Reciprocally, with an increase in temperature, the hydrophobic characteristic of copolymer is known to improve cell adhesion, as a result of increasing of non-specific binding of adhesive cell matrix proteins with hydrogel.

Based on these results, we sought to modify the hydrogel properties. A range of thermo-responsive formulations were synthesized and characterized to optimize the molecular weight, LCST, aqueous solution concentration, and viscoelastic properties. These included homo-polymers of PNIPAM, co-polymer of NIPAM with N-tert-butylacrylamide (NT), and co-polymer of NIPAM with butylacrylate (BA), each using NIPAM as the main component of the formulation (Table 2).

TABLE 2
Copolymer formulations

| | | PNIPAM-co-N-tert- | PNIPAM-co- |
|---|---|---|---|
| | | Butylacryamide | Butylacrylate |
| | PNIPAM | (NₓT_{y}) | (NₓBA_{y}) |
| Chemical Formula | (C₆H₁₁NO)x | (C₆H₁₁NO)ₓ:(C₇H₁₃NO)_{y} | (C₆H₁₁NO)ₓ(C₅H₁₁O₂)_{y} |
| Co-Polymer Ratio | | (85:15) | (95:5);(88:12) |
| Molecular Weight | 20.0 × 10³ to 2.8 × 10⁵ | 5.5 × 10⁵ to 6.6 × 10⁵ | 30.0 × 10³ to 5.2 × 10⁵ |
| Aqueous Solution | 10% to 43% | 10% to 30% | 10% to 30% |
| Concentration | | | |
| LCST (° C.) | 32 | 25 | 15 to 25 |

*x and y are integers having values that achieve the indicated molecular weight.

The phase transition temperature is deliberately shifted of PNIPAM to lower temperatures to ensure a higher level of hydrophobicity in the hydrogel at eye temperature. In order to ascertain the contribution of the NT and BA monomers to the phase transition, scattering intensity measurements were carried out for aqueous PNIPAM, poly(NIPAM-co-N-t-butylacrylamide), N₈₅NT₁₅, and N₉₅BA solutions from 2-26° C., which includes the phase transition temperature (FIG. 2B). It was observed that scattering intensities at higher temperature (above phase transition temperature) were relatively larger than those at lower temperatures (below phase transition temperature), which is mainly due to the transformation from a more soluble coil conformation below the LCST to a largely insoluble compact conformation. For instance, the scattering intensity values of N₉₅BA₅ exhibited a sharp increase in the temperature range of 16° C., which is indicative of gelation formation point. But value of scattering intensity gradually decreases above 22° C., which implies the cluster sizes are big enough at 24° C. to impart translucence to the solution (FIG. 1G). Comparison of temperature-dependent scattering intensity distributions of three different stimuli-responsive hydrogels confirmed that PNIPAM shows a gelation formation point around 32° C. By inclusion of only 5% of BA or 15% of NT, the gelation formation point shifted to 16° C. and 22° C. respectively (FIG. 1G). We successfully synthesized and engineered smart hydrogels, N₈₅NT₁₅ and N₉₅BA₅, with appropriate phase transition temperature for human eyes (FIG. 2B).

Improved adhesion performance was explored by co-polymerizing pNIPAM with N-tert butylacrylamide (NT) and butylacrylate (BA). Therefore we invested significant effort in exploring two co-polymer formulations: poly(NIPAM-co-N-tert-butylacrylamide) and poly(NIPAM-co-butylacrylate) in an effort to improve adhesion performance. It is well understood that the addition of the second monomer to PNIPAM serves to lower the lower critical solution temperature (LCST). We deliberately shifted the LCST to lower temperatures to ensure a higher level of hydrophobicity in the polymer at body temperature. Table 2 is a summary of the range of chemistries and formulations tested.

Continued Development of Hydrogel Chemistries with Improved Performance

We continue to explore the development of different chemistries to further enhance the hydrogel adhesion to ocular tissue. We identified three categories of additives that can be integrated into the hydrogel polymer chemistry to further enhance the adhesion. A range of different additive concentrations have been prepared and tested in vitro using our previously describes IOP test model system.

Original IOP Testing System. Preliminary IOP measurements using cadaveric porcine eyes yielded wide variations in results when testing the same samples. Variations in the vitrectomy performed to prepare each eye, as well as, variations in freshness of tissue resulted in wide variations in measured IOP.

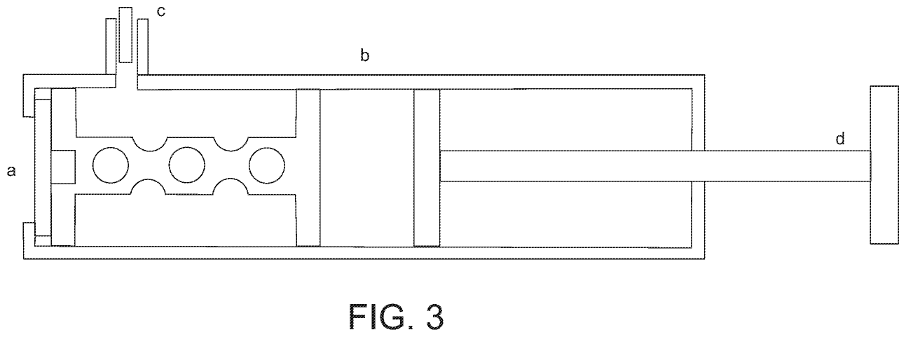

Revised IOP Testing System. A revised IOP testing system was designed that would allow us to repeatedly create similar penetrating injuries in scleral tissue, but which would also allow us to very carefully regulate the intraocular pressure to which the test specimen was subjected. FIG. 3 shows the design of the revised IOP Test system. Porcine sclera was dissected from the eyes used in the previous testing system, and mounted (a) in a modified 60 mL syringe (b). The syringe tip was machined to create an 8 mm diameter aperture where the scleral tissue was positioned. A modified plunger was created to fix the scleral tissue in place. The normal plunger (d) was then inserted behind the modified plunger. A small port was created on the sidewall of the syringe (c) to both load the syringe with heated phosphate buffered saline and to insert a digital pressure sensor to track IOP. IOP was controlled by connecting the syringe to a digital and automated infusion system (yellow device lower left), which allows pressure to be applied to the syringe plunger at a carefully controlled rate. This system allowed pieces of scleral tissue with varying sized/design penetrating injuries to be tested.

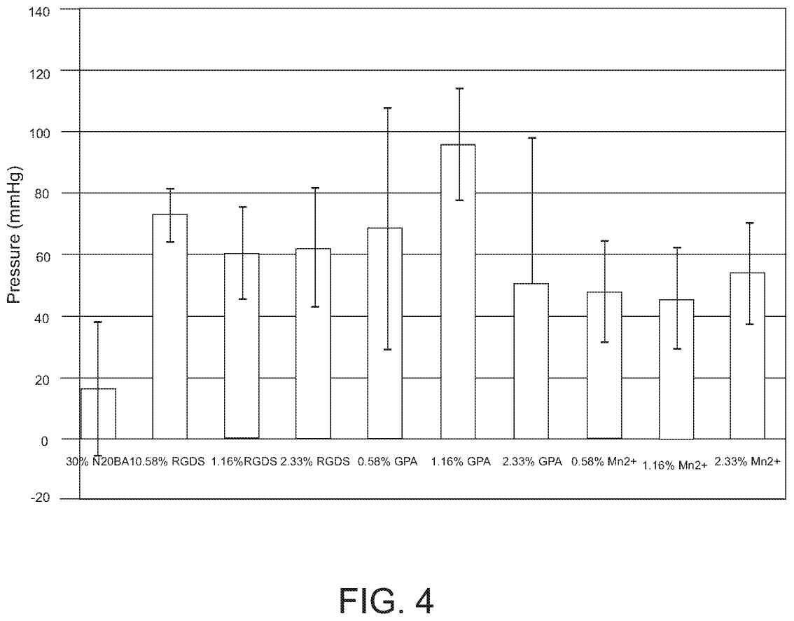

FIG. 4 plots the average IOP strength for three different hydrogel+additive mixtures tested with each additive tested at three different concentrations. For comparison, the bar plot at far left shows the average hydrogel-sclera adhesion for the co-polymer formulation using no additives. It clearly shows that 0.58% RGDS and 1.16% GPA failure pressure has improved 4.5 and 6 times more than no additive hydrogels respectively. These results suggest that the use of additives improve the adhesion strength of the hydrogel. We are moving toward preparing these samples for in vivo evaluation.

With respect to biocompatibility, the three additives tested do not raise major concerns. RGDS amino acid sequences are found naturally in many cell adhesion molecules and are commonly used in biomedical research. 3-guanidinopropionic acid is a common over-the-counter vitamin supplement. Manganese chloride is a common mineral salt and also used as a health supplement.

pNIPAM is a water soluble polymer whose aqueous solution exhibits phase transition at about 32, which is mainly due to transformation of its hydrophilic random coiled conformation to hydrophobic collapsed globular conformation. The precise conformation of macromolecule can be maintained by the monitoring of bonding interactions between macromolecule and solvent. As we know these interactions can perturb by changing temperature or by incorporating any third component to the aqueous solution of pNIPAM.

In some refinements, GPA is choses as a third component because it's functional groups are similar to Arginin in RGDS peptide, but it is very cheap and a common over-the-counter vitamin supplement.

DLS Measurement

The value of hydrodynamic diameter, R_{H}, was obtained by using the Stokes-Einstein equation:

$${R}_{H} = \frac{{k}_{B} T}{6 πη D}$$

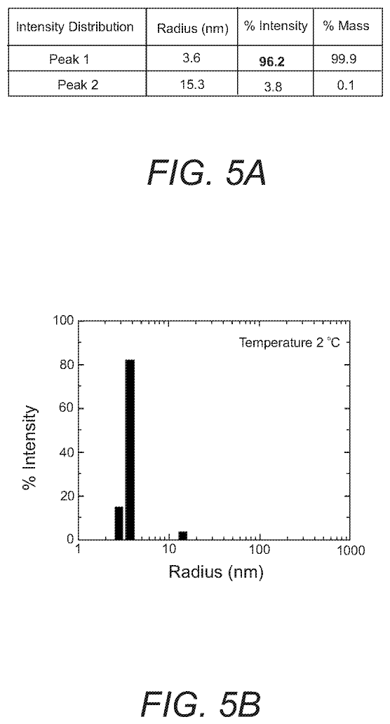

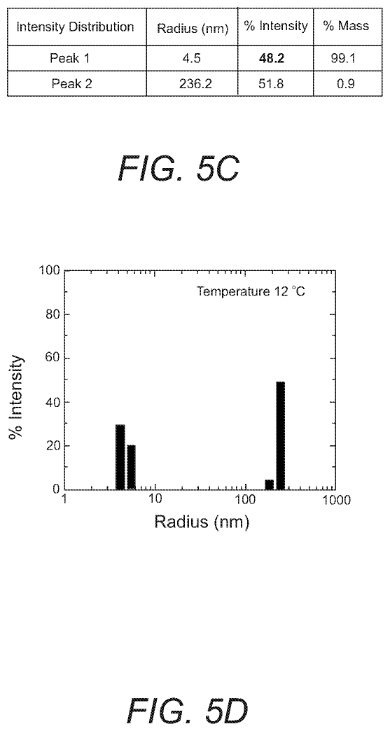

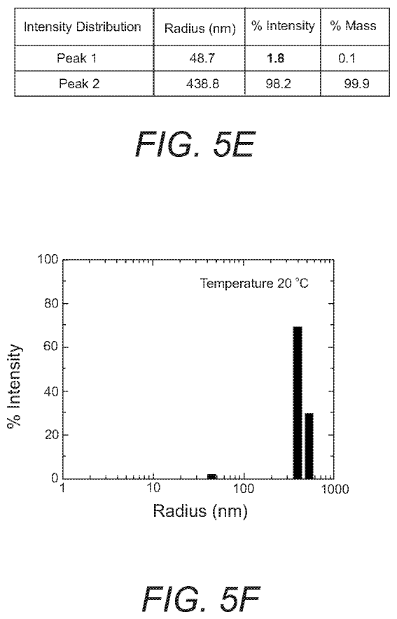

We employed DLS technique to investigate the effect of GPA hydrated states of copolymer in details. It is apparent that FIG. 5 displays intensity distribution of 5% N20BA1 at different temperatures. Below the phase transition temperature at 2° C., we observed that 96% of intensity corresponds to particles with a very small hydrodynamic radius (3.6 nm), which can be attributed to polymer in coil conformation (FIG. 4A). At the phase transition region, we observed two peaks for the sample. One of these peaks was obtained from a major population (490 nm) of high intensity. This peak shows that the N₉₅BA₅ molecules are highly aggregated in the solution. The other population (the second peak, 5 nm) corresponds to a lower percentage of intensity (Fig.). Further increase of temperature to 18° C. causes 98% of the copolymer to form large aggregates (436 nm), a consequence of more favorable polymer-polymer interactions. Amide groups facilitate these interactions by forming hydrogen bonds between polymer molecules, nesting themselves inside the globules.

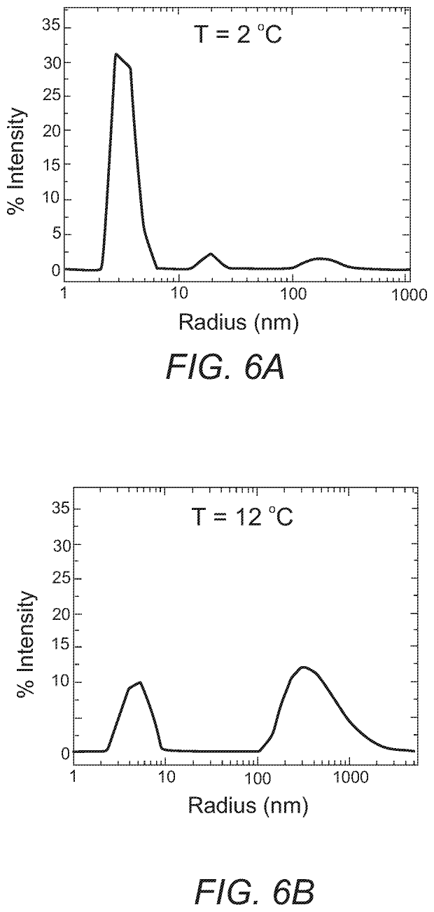

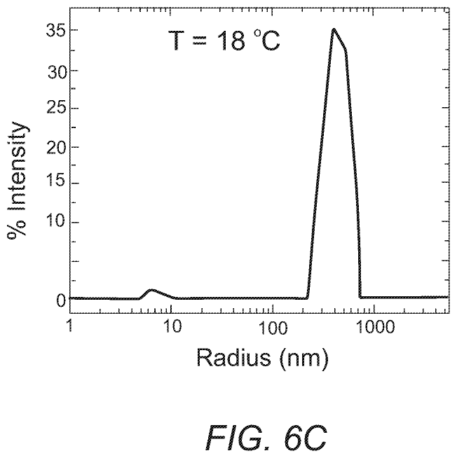

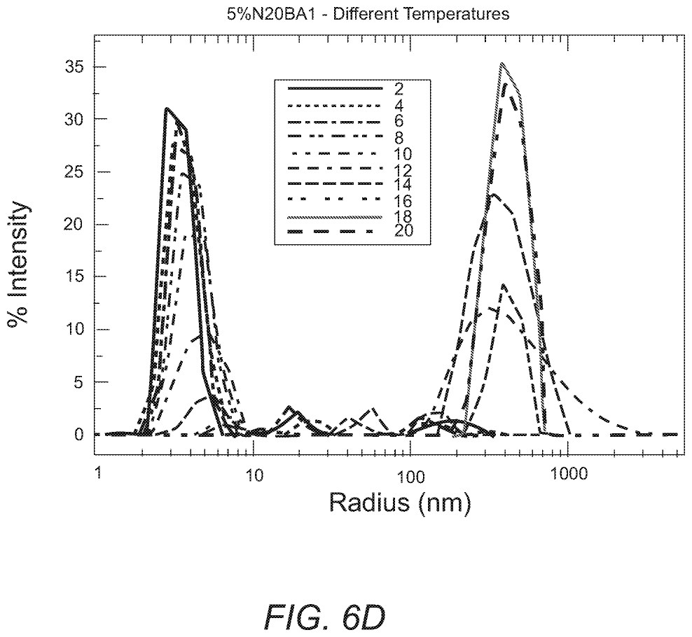

FIG. 6 shows intensity distribution of 5% N20BA1 in the whole range of temperature. As can be seen from FIG. 6, the R_{H} and intensity values of the first peak for smaller particles, are increasing and decreasing, respectively, with enhancement of temperature. But the values R_{H} of and intensity of the peak area for high aggregation, will increase as a result of heat induced aggregation.

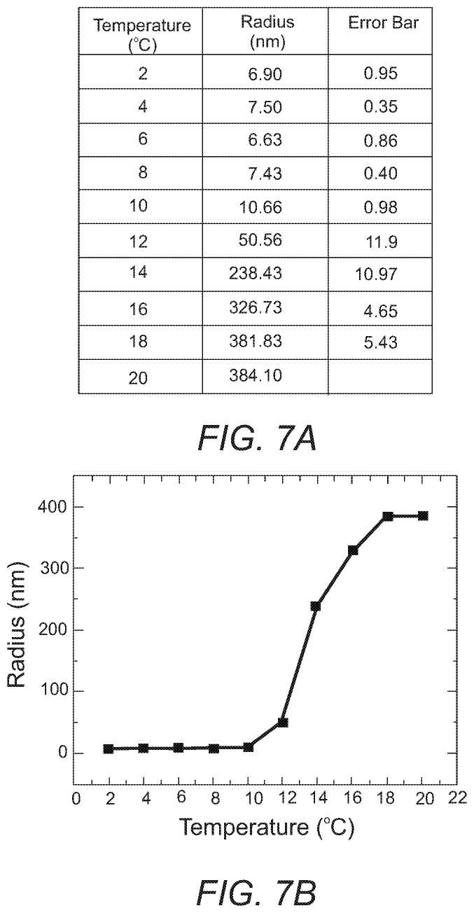

The temperature induced hydrophobic collapse of 5% N20BA1 in the absence of third component monitored in the whole range of temperature, including phase transition temperature, from DLS measurements. FIG. 7 apparently implies that even in the absence of additive, copolymer is capable of exhibiting heat induced aggregation. At the phase transition temperature this copolymer shows a clear phase transition upon heating, going from soluble to insoluble in the aqueous solution. As shown in FIG. 7, remains nearly constant (8 nm) up to 10 C and further increase of temperature, it tends to form large aggregations (490 nm) at 20 C as a consequence of reversible phase transition.

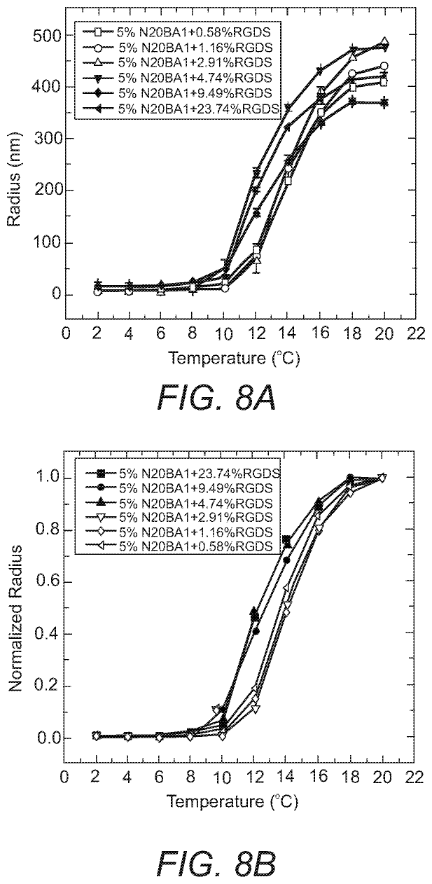

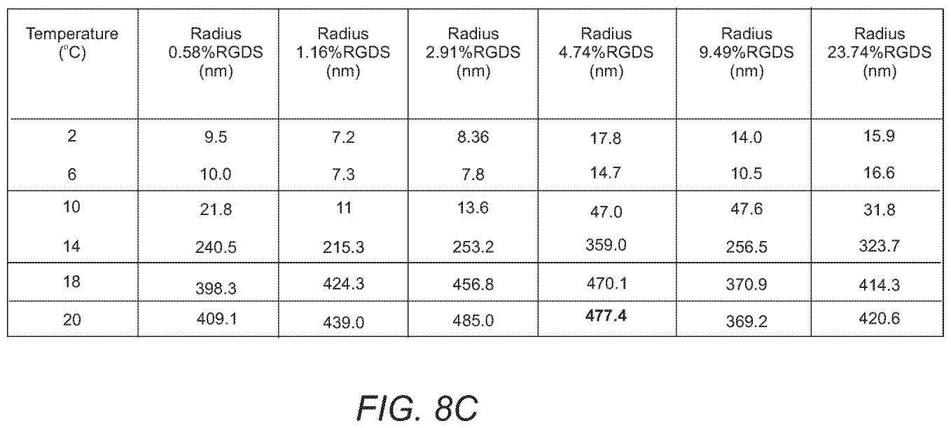

To ascertain the contribution of RGDS peptide on the phase transition and particle size of poly (N-isopropylacrylamide-co-butylacrylate) aqueous solution, we have further exploited DLS measurements as a function of RGDS concentration at various temperatures. FIG. 8 reveals the values of R_{H} as a function of temperature for RGDS in 0.58%, 1.16%, 2.91%, 4.74%, 9.49% and 23.7% mg/mg N20BA1. At temperatures below the LCST, we observed R_{H} was small (7.2-17.8 nm) at 8-10 C for various concentrations of RGDS, which can be attributed to polymer in coil conformation. It is remarkable that hydrodynamic radius rapidly increases around 12-18 C. Moreover, the increase of the size occurs in a quite wide temperature range (5-6 C) due to polymer starts globule state from coil conformation, revealing the phase transition of copolymer occurs in this region.

FIG. 8 explicitly elucidates the LCST region of N20BA1 copolymer aqueous solution is altered as a function of RGDS concentration. It can be seen that organic RGDS decreases the phase transition start's point (from 12° C. in the low concentration of RGDS to 10° C.) with increasing RGDS concentration (from 0.58% to 4.74%, 9.49% and 23.7%). Through this, we have explicitly found that the RGDS decreased the phase transition of copolymer solution, due to collapsing and aggregating the macromolecule.

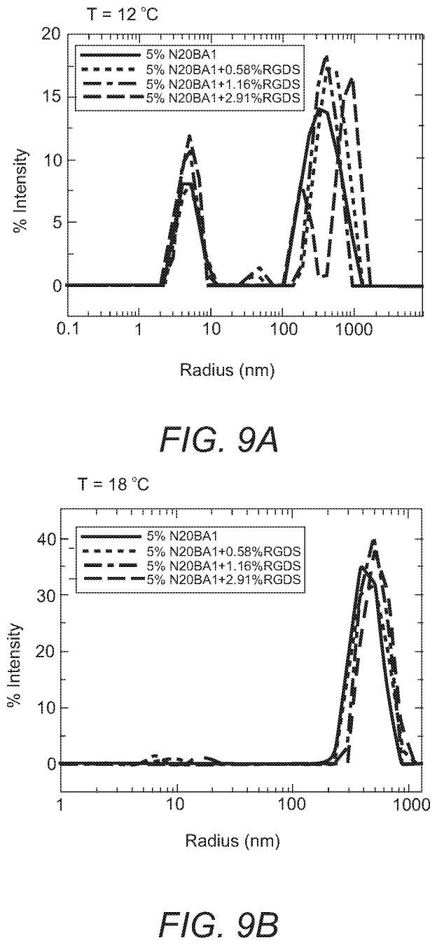

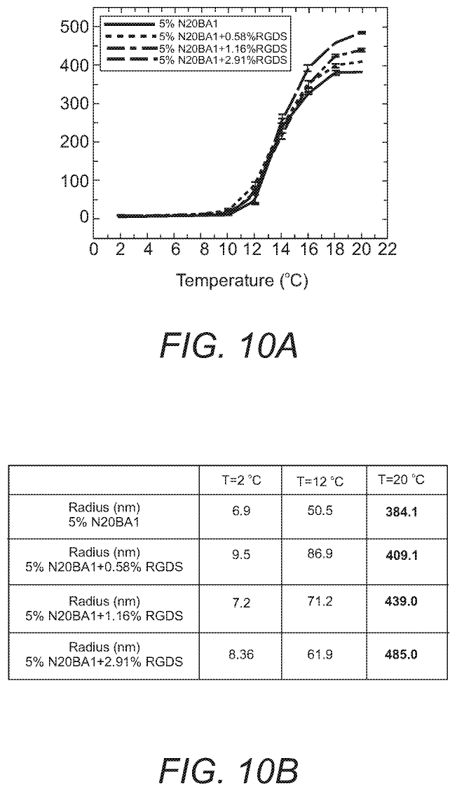

In the presence of RGDS, the copolymer chains start to aggregate substantially in the solutions. The particle size distribution graphs of aqueous copolymer solution with various concentration of RGDS is shown in FIG. 9. At 12° C., It clearly implies the existence of two sets of aggregates: one in the nano range and another in the range of 50-90 nm. Fraction of major population (70 nm) revealed an increase in size with temperature and RGDS concentration. Invariably, for all the samples, at 12° C. the size of aggregates increased typically by 7-fold or more. The variation of the Rh of N20BA1 aggregates as a function of temperature and RGDS concentration is shown in FIG. 10. With an increase in the concentration of RGDS, there was an increase in the size of the aggregate. The Rh of these aggregates increased with temperature too. Due to the significant gain in turbidity of the copolymer solution, DLS measurements could not be performed above 20° C. Below the LCST, the Rh of RGDS samples increased by small amount. Above the transition temperature, 2.91% RGDS increased polymer's particle size around 100 nm.

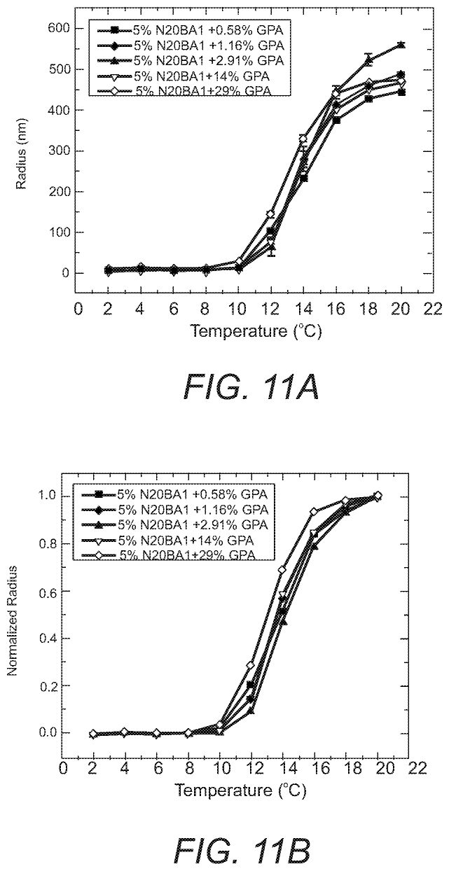

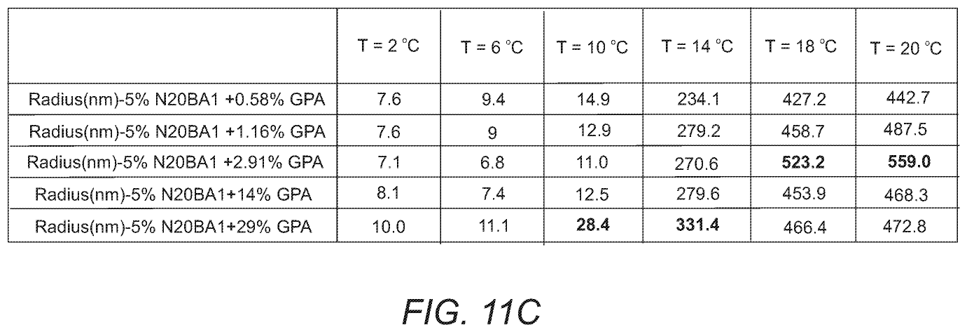

FIG. 11 depicts the temperature dependent growth of aggregates. With increasing concentration of GPA, there is an indication of enhancement in the size of the aggregates. The hydrodynamic radii, (Rh) values of these aggregates increase with temperature. It was observed that the nanometric copolymer aggregates in the solution state start to grow at the temperature of gelation-onset. Moreover this onset temperature did not change as GPA concentration increased. In the presence of GPA, N20BA1 copolymer chains start to aggregate forms larger particles, and with increasing temperature the size of the aggregates further increased due to the hydrophobic interaction of pNIPAM chains. As can be seen from FIG. 11, the Rh values of GPA contained samples seem to follow the same trend as RGDS. All GPA contained hydrogels give a gelation-onset temperature at 12° C.

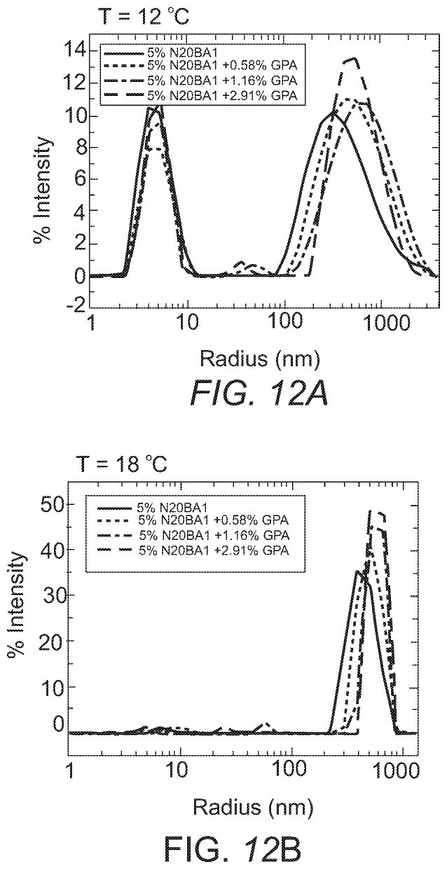

FIG. 12 shows the GPA concentration-dependent variation of intensity for N20BA1 aggregates at two different temperatures of 12° C. and 18° C. The intensity of small particles (8 nm) decreases gradually over the temperature rage 12-18° C. Again, the size distribution curve (FIG. 12) for N20BA1 in (0.58%-2.91% w/w) GPA shows a similar trend as in pure N20BA1 solution. Since these measurements were made at the same temperature, the changes in the size particles should not be due to intra- or intermolecular hydrogen bonding between polymer segments. Therefore, this change in the size particles arises from the interaction between the polymer and GPA molecules. It is also noted that, in the intensity distribution graph, the peak area for high aggregation will appear at least 55 times larger than that of the first peak for smaller particles.

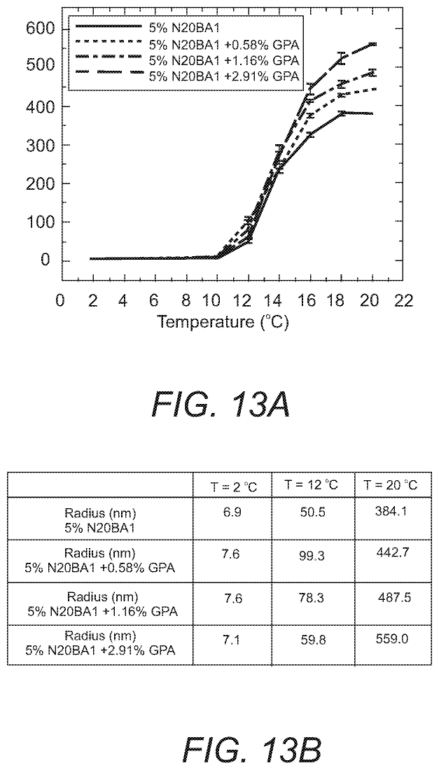

FIG. 13 shows the variations in hydrodynamic radius values of copolymer in aqueous solution in the absence and presence of different concentrations of GPA. We noticed that the onset of Rh values increment was shifted toward lower temperature in the presence of all GPA contained samples. FIG. 13 implies that even in the presence of GPA, phase transition temperature did not change. One can clearly see from FIG. 13 that even a low concentration of GPA is sufficient to influence the polymer conformation.

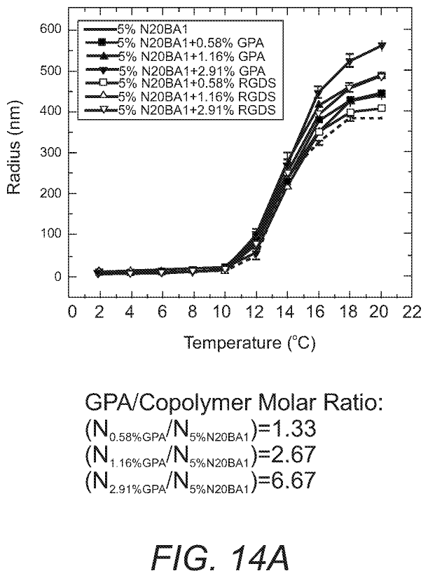

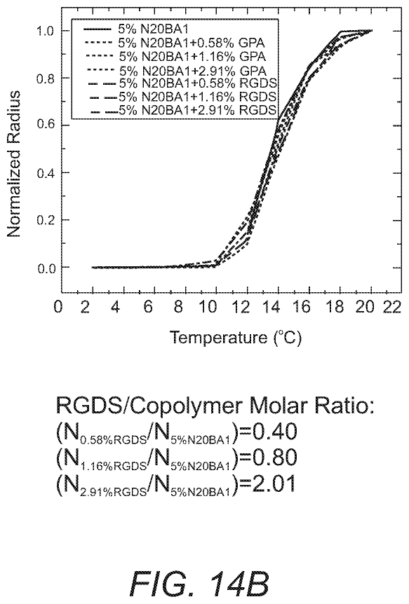

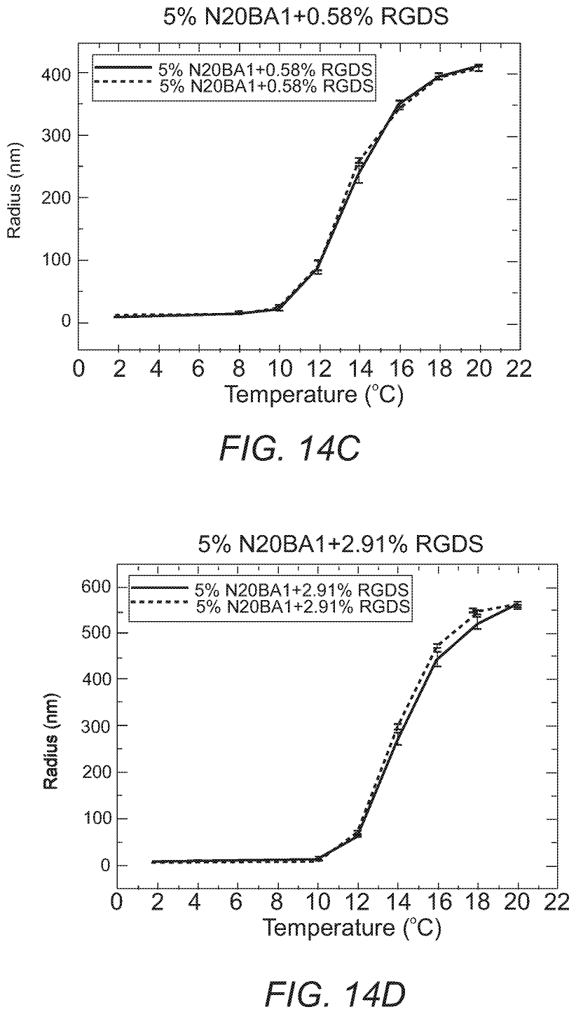

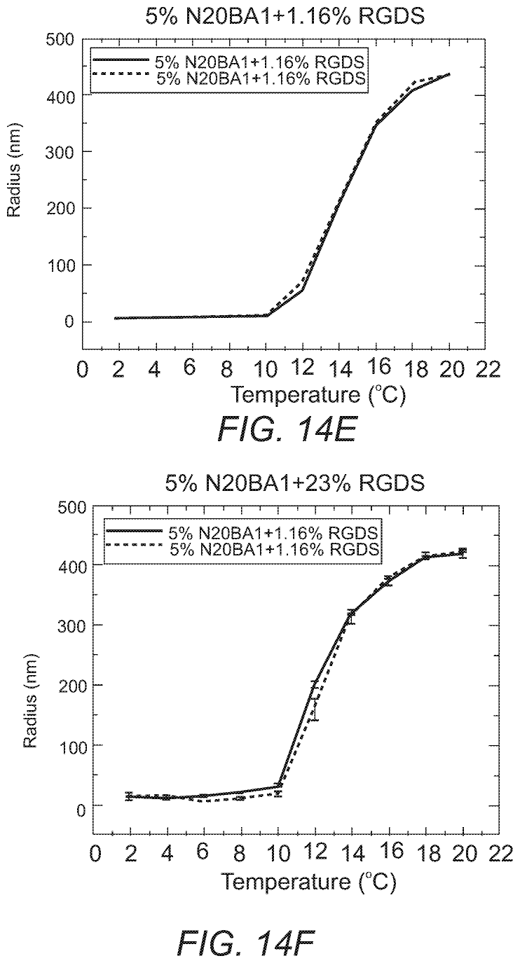

A temperature-dependent variation of hydrodynamic radius in the presence of RGDS and GPA was shown in FIG. 14 and it clearly dictates that the values of Rh increase with enhancement of RGDS and GPA concentration. Interestingly, marked increase of Rh values in additive-included samples were found at phase transition temperature of additive-free hydrogel, over the whole range of the present concentrations of RGDS and GPA in FIG. 14. Moreover, the phase transition temperature values of additive-included hydrogels are almost as same as additive-free aqueous N20BA1 solution. It clearly represents that, at low concentrations of RGDS and GPA, it is insufficient to influence the polymer conformation by rupturing the hydrogen bonds between polymer and water molecules. However, the phase transition temperature values changed at higher concentrations of RGDS (FIG. 8).

To further investigate the effect of third component on hydrated and dehydrated states of N20BA1 copolymer in details, we calculated the molar ratio of additives to copolymer. It was verified that values of Rh are almost the same for similar molar ratio. For instance, 1.16% GPA (molar ratio of 2.66) shows the same Rh value as 2.91% RGDS (molar ratio of 2.01) at 20° C.

Fluorescence Studies:

All fluorescence measurements were performed using a Cary Eclipse Fluorescence spectrophotometer with an intense Xenon flash lamp as the light source. The sample solutions was introduced into the quartz cuvette with the help of micropipette. The sample containing quartz cuvette was placed in a multicell holder, which was electro-thermally controlled at precise temperature regulated by peltiers.

In the present study, 8-anilino-1-naphthalene-sulfonic acid (ANS) was used as a fluorescence probe. ANS gives a very weak intensity at 510 nm in aqueous solution. However, ANS shows either a blue shift or red shift depending upon the decrease or increase in local polarity and mobility.

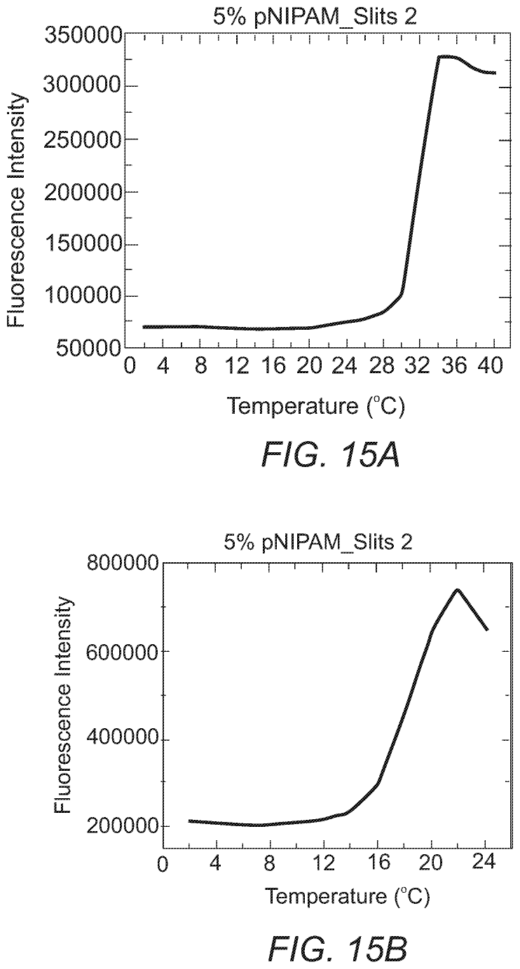

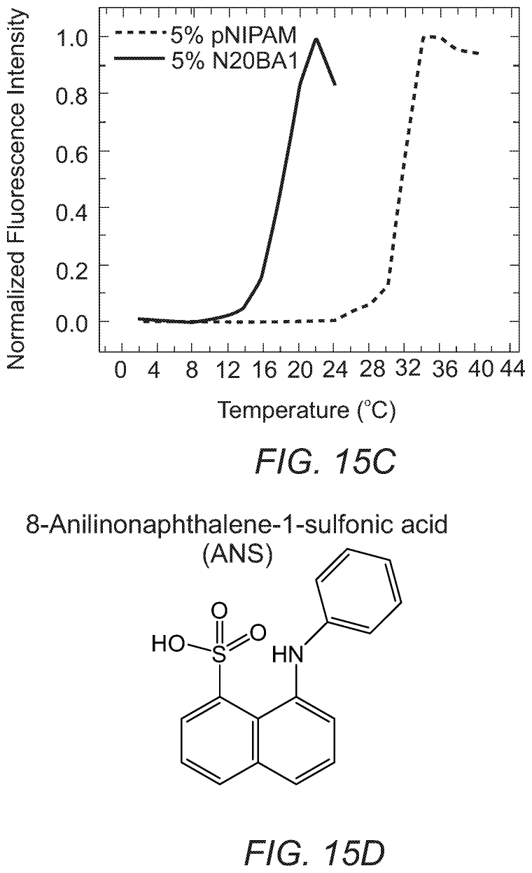

For the current study, the profile of the fluorescence emission spectrum of the probe was recorded upon the temperature of copolymer aqueous solution. As shown in FIG. 15, at higher temperature, the intensity of the probe was higher than that at lower temperature. It symbolizes that the polymer adopts a relatively compact globule conformation at higher temperature, whereas below the LCST the polymer exhibits a coil conformation. From FIG. 15, one can easily understand that the pNIPAM homopolymer exhibits a higher intensity around 34° C., whereas the poly (NIPAM-co-butylacrylate) shows a higher intensity approximately at 22° C. Clearly it indicates that the LCST values decrease around 12° C. by only adding 5% butylacrylate.

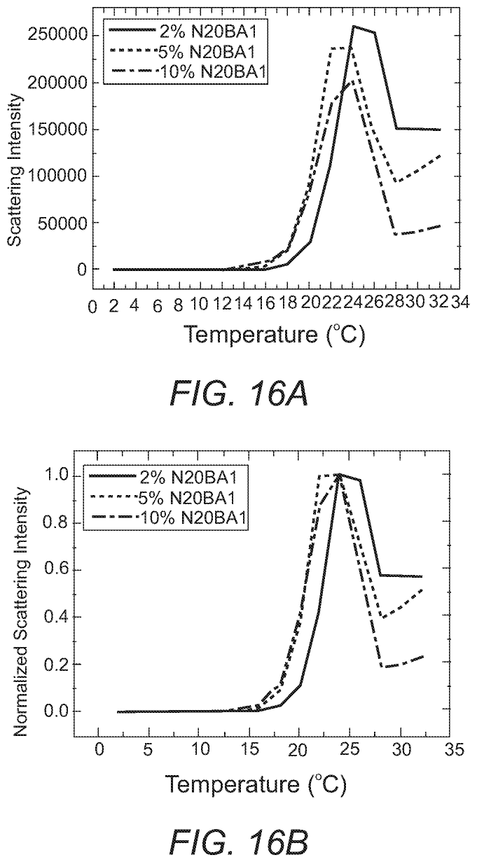

FIG. 16 shows a graphical representation of copolymer concentration-dependent scattering intensity spectra. The graph of scattering intensity shows that by increasing the concentration of copolymer the intensity maximum moves to toward lower temperature.

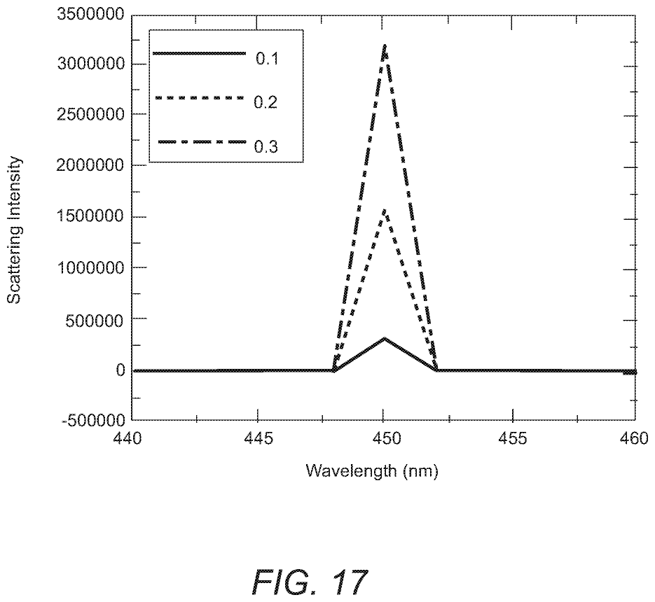

Emission spectra were recorded with different slits width in order to have a better understanding to choose appropriate slit for 5% N20BA1 sample, and results are shown in FIG. 17.

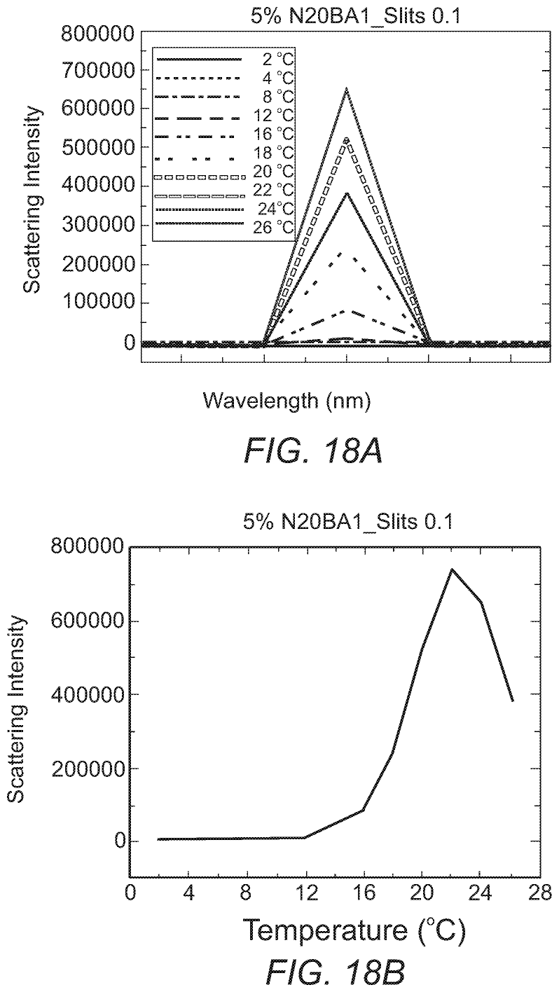

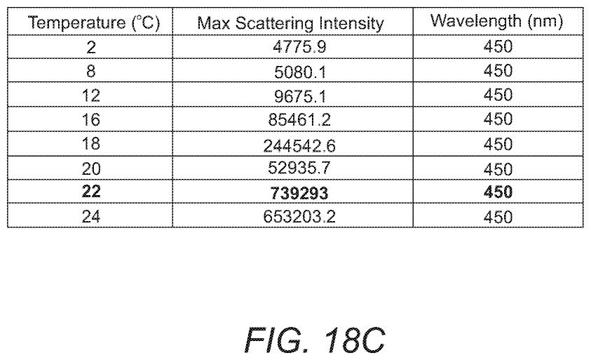

Temperature dependent scattering intensity measurements were exploited for aqueous N20BA1 solution, and the results are shown in FIG. 18. It clearly shows that intensity at higher temperature (above phase transition temperature) was relatively high than that of lower temperatures (below phase transition temperature), which is mainly contributed from the transformation of well soluble compact conformation (at low temperatures) to sparingly soluble compact conformation (at higher temperatures). FIG. 18 shows that the value of scattering intensity gradually decreases, which implies the cluster sizes are big enough at 24° C. to impart translucence to the solution.

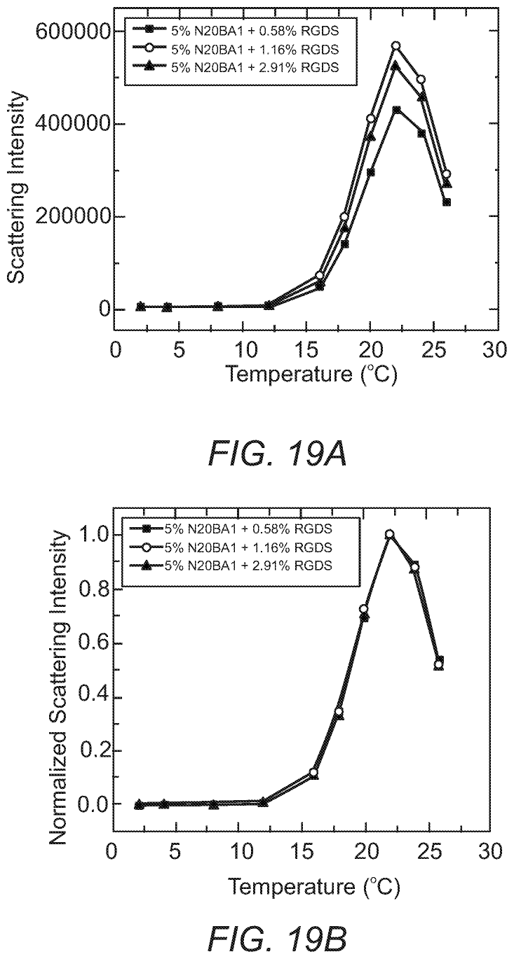

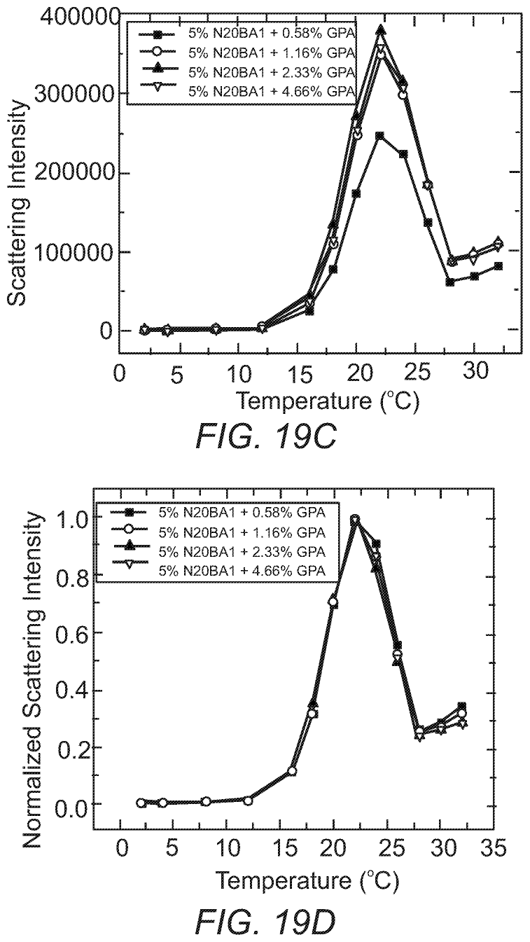

The response of copolymer scattering intensity to the increasing concentration of RGDS and GPA in aqueous media at different temperature is shown in FIG. 19 As can be seen from FIG. 19, for all solutions there is an increase in scattering intensity with increasing temperature. Since copolymer forms a turbid gel above its phase transition temperature, the scattering intensity decreases. Increase in the additive concentration leads to increased scattering intensity, but the intensity maximum's temperature does not change, closely following the trend of DLS results. A large enhancement of scattering intensity was observed in 5% N20BA1-1.16% RGDS and 5% n20BA1-1.16% GPA media which indicates RGDS and GPA-induced structural changes in copolymer. The scattering maximum observed at 22° C. for all additive-containing solutions.

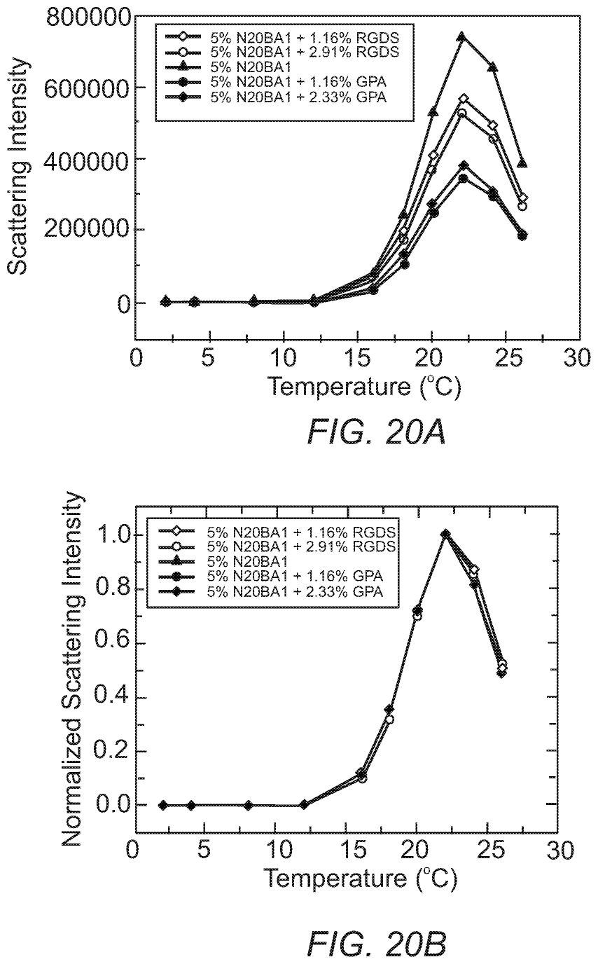

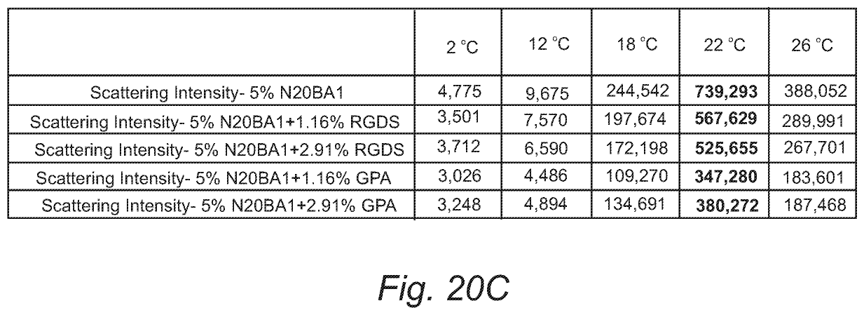

FIG. 20 shows the scattering intensity of N20BA1 in the presence and absence of RGDS and GPA as a function of temperature. The N20BA1 concentration was 5% (w/v), and additive concentration was varied from 0 to 2.91% (w/w). As shown in FIG. 20, during the heating process of solutions, the sharp enhancement in intensity was observed at 12° C. for all the samples. The observed sharp enhancement in intensity was an indication of gelation formation. Below the gelation formation temperature (GFT), the scattering intensity values in additive-containing solutions are similar to that for pure copolymer solution. Whereas, above GFT, the free additive sample exhibits a higher scattering intensity comparing to additive-containing solutions. This indicates that RGDS and GPA does play a significant role in inducing larger aggregation of copolymer. Moreover, the scattering intensity are relatively higher in the presence of RGDS than in the presence of GPA.

Viscosity Measurement:

Viscosity measurement was carried out as a function of temperature with a sine-wave viscometer. Temperature was regulated with a circulating water bath. Viscosity data was measured to study the conformations of copolymer in aqueous solution with or without addition of RGDS and GPA in the temperature range from 2 to 40° C.

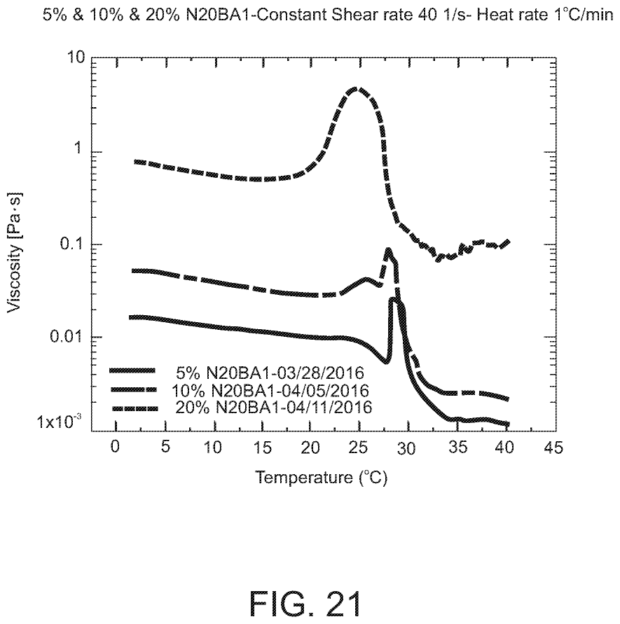

FIG. 21 depicts viscosity curves of different concentrations of poly (NIPAM-co-butylacrylate) solution in the absence of additive as a function of temperature. It is clearly illustrated from FIG. 21 that aqueous N20BA1 solutions have high viscosity at temperatures lower than its phase transition temperature due to strong hydrogen bond formation between the amide group in the macromolecule and water molecules. On the other hand, with increasing temperature, the copolymer becomes dehydrated and it would collapse and takes to the compact globule structure; therefore, the viscosity values gradually decrease and finally approached a constant value. FIG. 21 also shows that the viscosity of N20BA1 solutions increase sharply in the temperature range of 27-29° C. (5% N20BA1) which is indicative of gelation formation point. As can be seen from FIG. 21, increasing concentration of copolymer significantly increases the viscosity values in the whole range of temperature. These results indicate that, the higher concentration of copolymer (20% N20BA1) is able to affect the LCST and viscosity of polymer solution by adsorbing more water molecules and making more hydrogen bonds between the amide group and water molecules.

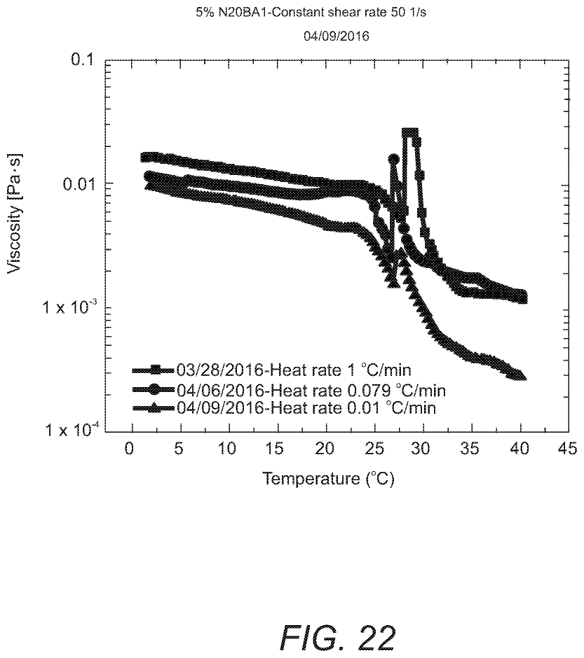

To investigate the effect of kinetic on the viscosity of 5% N20BA1 (w/v), we performed viscosity measurements at different heat rates. From FIG. 22, it was observed that the nanometric N20BA1 aggregates in the solution state start to grow at the temperature of gelation-onset, where there is a sudden increase in viscosity values. A possible explanation of the abrupt increase of viscosity could be as follows. When viscosity measurements of the sample were taken at a fast heating rate (1° C./min), the sample does not have enough time to achieve thermodynamic equilibrium. The results clearly show that by changing the heating rate to smaller value (0.01° C./min), the sharp shift in viscosity value and the onset temperature decrease, as expected.

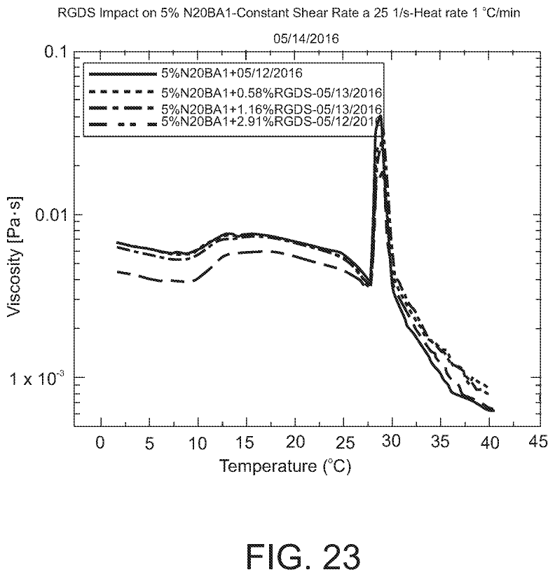

The viscosity values of aqueous N20BA1 (5% w/v) solution in the presence of RGDS were measured over a broad range of temperature. FIG. 23 depicts the temperature and RGDS concentration dependent viscosity of copolymer aqueous solution. It is clearly illustrated that viscosity of RGDS-free aqueous copolymer solution (indicated in black line) is about 8 mPa·s at 15° C. By increasing the temperature, the viscosity was decreased gradually up to 27° C. (GFT) and then a sharp increase was observed (see FIG. 22) and eventually, it decreased with further enhancement of temperature. From FIG. 23, it is clear that below GFT, the viscosity value decreases with increasing RGDS concentration. Interestingly, the sudden increase in viscosity (GFT) was found approximately at 27° C. for all types of solutions. The maximum and minimum extent of reduction in viscosity was observed in the presence of 2.91% and 0.58% RGDS-containing samples, respectively.

During the aggregation process, the larger aggregates coalesce to form microdroplets that undergo Ostwald ripening and eventually sediment out of the solution. Thus, the data presented in FIG. 23 mostly owe their origin to the smaller aggregates. However, these aggregates grow in size with an increase in temperature, and beyond 30° C., these aggregates become sufficiently large to remain in a stable suspension. Loss of these big aggregates to sedimentation may manifest itself in the reduction of the viscosity of the solution beyond 32° C.

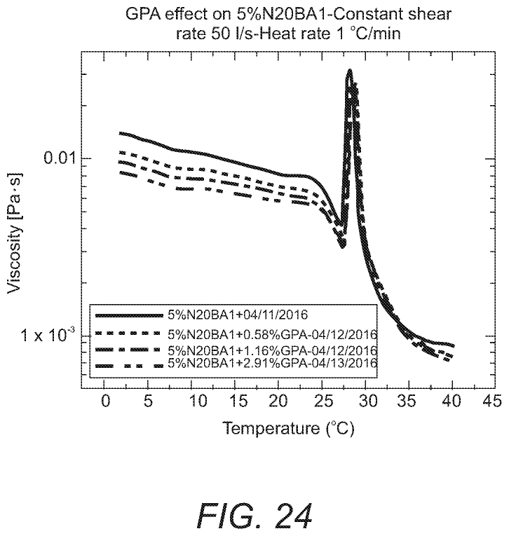

In FIG. 24, temperature dependent viscosity of N20BA1 (5% w/v) aqueous solution (black color line) and in the presence of different GPA concentrations, are shown. As one can clearly observe, the viscosity curves follow the same trend as RGDS-added samples (see FIG. 23). Three different regions were observed in viscosity plots as copolymer transformed from expanded unimers (at low temperatures) to aggregates (at high temperatures). At low temperatures (<GFT), the solution has larger viscosity values due to hydrated copolymer chains. However, at higher temperature (>LCST), the copolymer become dehydrated and it would form the core and takes interior portion of micelle; therefore, the solution shows lower viscosity. This indicates that the hydrogen bonds between copolymer amide group and water molecules are ruptured at higher temperatures, allowing hydrophobic interactions that occur between the segments of copolymer chains.

Moreover, viscosity value of N20BA1 solutions ware low in the presence of GPA comparing with viscosity of additive-free copolymer aqueous solution. This observation can be interpreted that the entire series of GPA-added hydrogels are good enough to rupture the hydrogen bonds between copolymer and water molecules, and thereby make hydrogen bonds with copolymer.

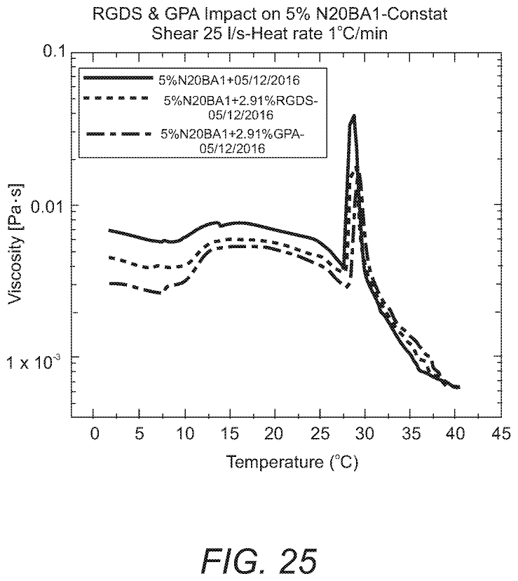

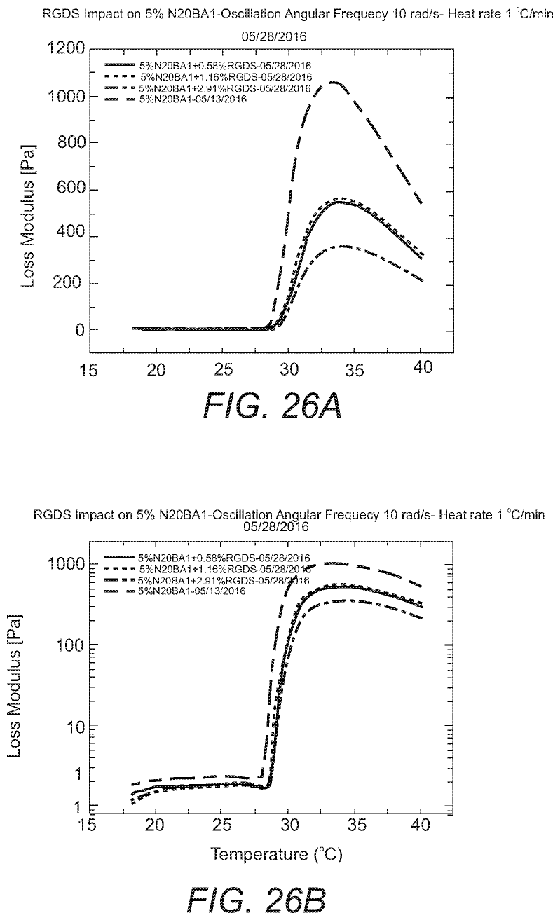

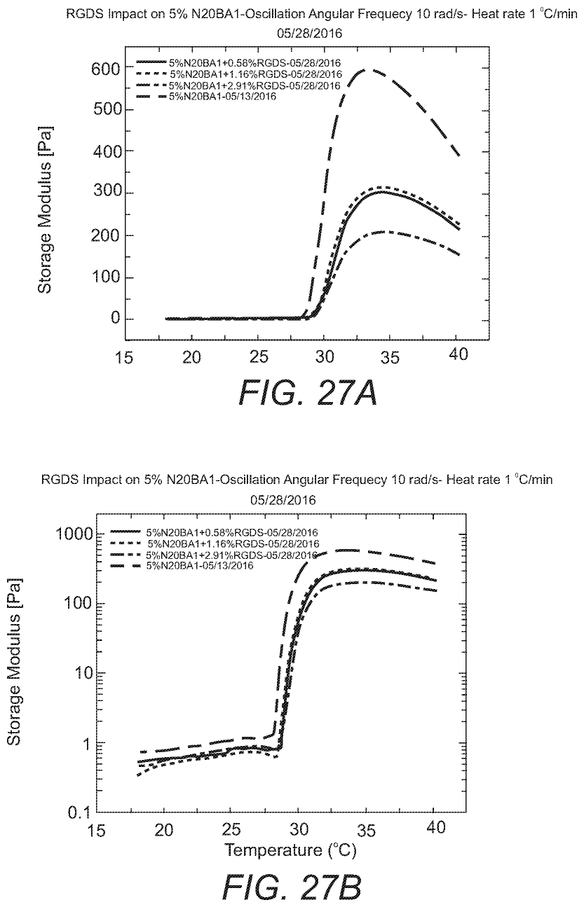

From the previous figures, it was identified that RGDS and GPA play a significant role in decreasing viscosity of aqueous poly (NIPAM-co-butylacrylate) solution in the temperature range of 2-30° C. (see FIGS. 23 and 24). To compare effect of RGDS vs. GPA on the free additive copolymer, we have explored the viscosity values of N20BA-2.91% RGDS, N20BA1-2.91% GPA and N20BA1 solutions in the whole temperature range of 2-40° C., and the results are shown in FIG. 25. It was observed from viscosity measurements, that the onset of the LCST of copolymer solution does not change in the presence of RGDS and GPA. On the other hand, even small fraction of GPA can influence the viscosity of the copolymer significantly. However, at low concentration of RGDS, it is insufficient to influence the viscosity values of aqueous copolymer solution. This can be rationalized in the following way: as increasing the concentration of RGDS, the interaction between copolymer and water decreases, and thereby, we observed a shift in viscosity to smaller values.

The Sclera mainly consists of collagen fibers and proteoglycans embedded in an extracellular matrix.

Recent reports indicate that the permeability of drug molecules across the sclera is inversely proportional to the molecular radius. Furthermore, the charge of drug molecule also affects its permeability across the sclera. Positively charged molecules exhibits poor permeability presumably due to their binding to the negatively charged proteoglycan matrix.

Polystyrene nanospheres do not diffuse freely into the vitreous due to their adherence to collagen fibrillary structures.

Vitreous is a greatly hydrated (98% water) transparent biogel consisting mainly of collagen (_300_g/mL), hyaluronan (65-400_g/mL), and proteoglycans containing chondroitin sulfate and heparan sulfate. Besides these components, vitreous contains noncollagenous proteins and serum components and low numbers of hyalocytes. The gel-like behavior of vitreous arises from an ordered, 3-dimensional network of collagen fibrils bridged by proteoglycans filaments. Hyaluronan is known to bind the chondroitin sulfate part of proteoglycans and to fill up the interfibrillar spaces.

It is clear from FIG. 3 and photolysis experiments that the polystyrene nanospheres strongly bind to the biopolymers in the vitreous. Given that both the polystyrene nanospheres and the biopolymers in the vitreum are negatively charged, we speculate that the nature of this binding is not electrostatic but rather hydrophobic. In addition, because collagen mainly consists of the hydrophobic amino acids glycine, proline, and hydroxyproline, we hypothesize that the fibrils to which the polystyrene nanospheres bind are collagen fibrils.

Results for Two Step Methods Using a Primer Layer

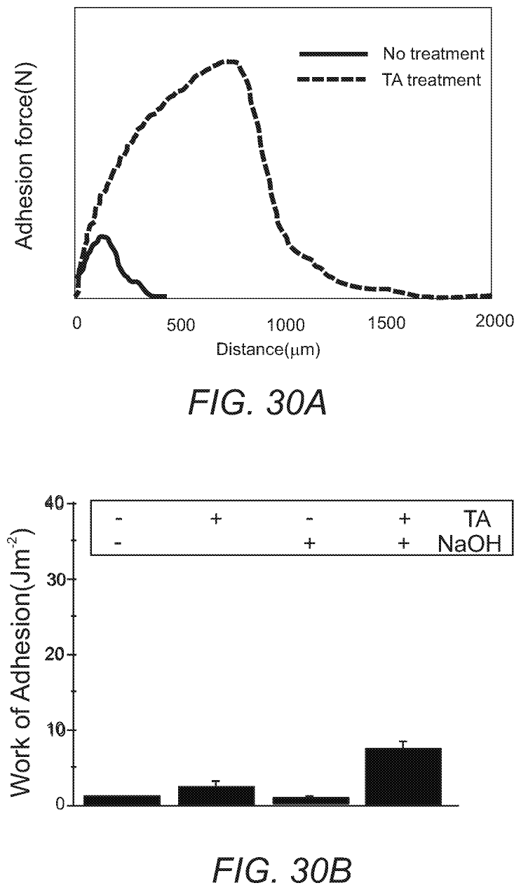

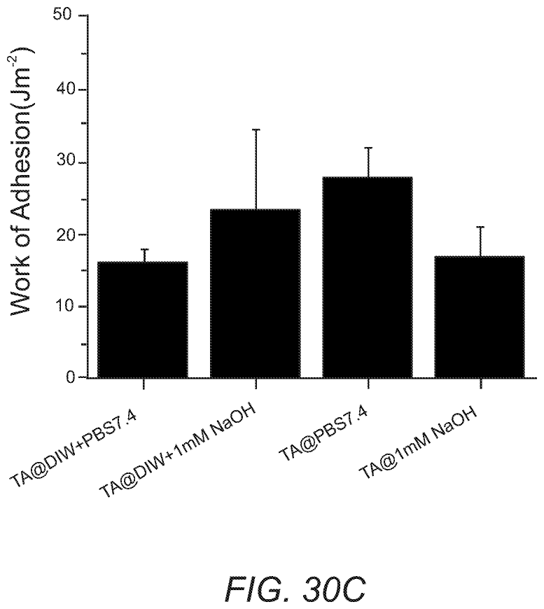

The adhesion performance of P(NIPAM₁₀₀-BA₅) to porcine sclera is greatly improved using tannic acid (TA) as the priming layer. The adhesion strength is optimal when TA is applied followed by the increase of the surface pH. The deployment method of TA solution can be varied, either using a two-step method with TA dissolved in deionized (DI) water plus weakly alkaline water and rinsing with DI water after each step, or using a one-step method with TA dissolved in alkaline water and rinsing with alkaline water afterwards. FIG. 30 provides a comparison of the adhesion strength of P(NIPAM100-BA5) matrix to porcine sclera using different deployment methods.

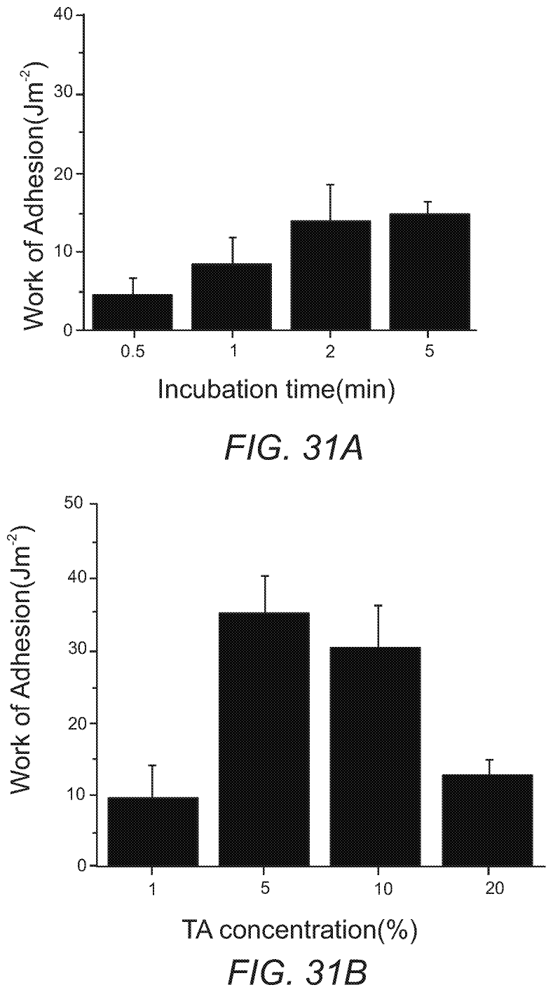

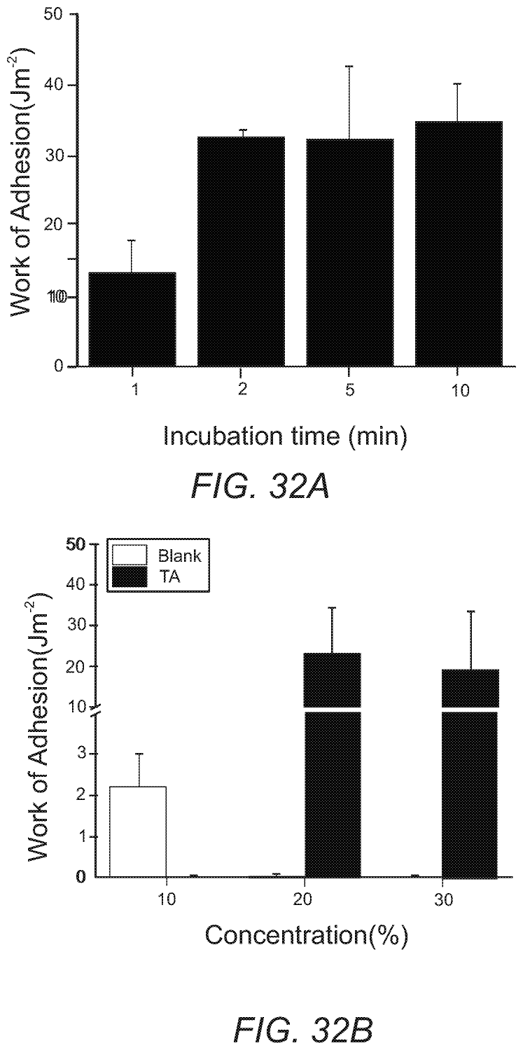

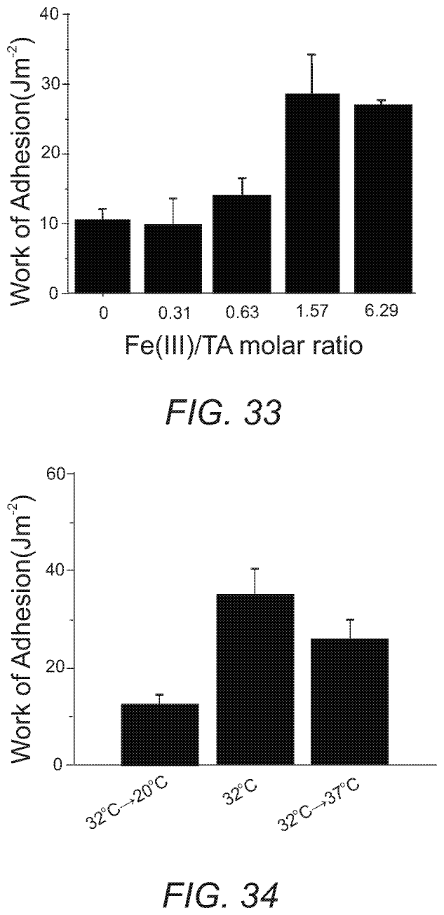

The adhesion strength can be varied by changing the TA concentration and the incubation time. FIG. 31 shows that the adhesion strength of P(NIPAM100-BA5) matrix to porcine sclera can be changed by varying the TA concentration and incubation time. The adhesion strength can be further varied by changing the polymer concentration and the incubation time as demonstrated in FIG. 32 which changes adhesion strength of polymer matrix to porcine sclera by varying the P(NIPAM₁₀₀-BA₅) concentration and incubation time. The adhesion strength can be further increased by using a mixed solution of TA and Fe(III) ion. Coordination of TA with Fe(III) can enhance the inter-molecular interactions. In this regard, FIG. 33 shows that adhesion strength of P(NIPAM₁₀₀-BA₁₀) matrix to porcine skin can be changed by varying the Fe(III)/TA molar ratio.

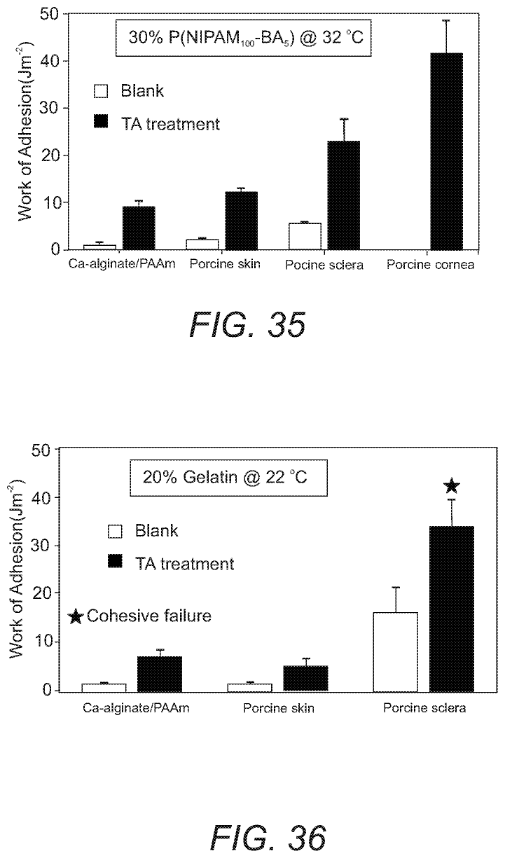

The polymer adhesive can be released easily by lowering the temperature, causing a great loss of adhesive strength. In this regard, FIG. 34 illustrates the adhesive strength of P(NIPAM100-BA5) matrix to porcine sclera using TA priming layer tested at different temperatures. As shown in FIG. 35, Using TA priming layer, the polymer matrix shows strong adhesion to different substrates including porcine skin, cornea, sclera and Ca-alginate/polyacrylamide hydrogel. FIG. 35 provides validation of adhesion enhancement of P(NIPAM100-BA5) matrix to different substrates using TA priming layer. Finally, the TA priming layer method can be extended to other thermoresponsive polymer matrix such as gelatin gels, and an increase of adhesion strength is observed on different substrates compared with the ones without TA priming layer. FIG. 36 provides validation of adhesion enhancement of gelatin gels to different substrates using TA priming layer.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A temperature-responsive hydrogel comprising:
water;
a poly(N-alkylacrylamide) copolymer of a first monomer having formula 1 and a second monomer that is different than the first monomer: 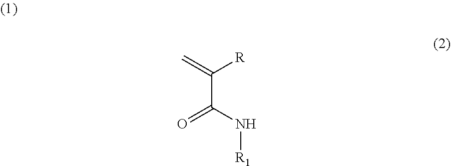
wherein:
R is H or C₁₋₆ alkyl;
R₁ is —(CH₂)ₙ—R₃, C₁₋₆ alkyl, C₆₋₁₈ aryl, or C₄₋₁₈ heteroaryl;
R₃ is H, hydroxyl, F, Cl, Br, NH₂, or N(R₄)₂;
R₄ is H or C₁₋₆ alkyl; n is an integer from 0 to 6 (i.e., 0, 1, 2, 3, 4, 5 or 6) and
X is O or NH; and
an adhesion-enhancing additive selected from the group consisting of Arg-Gly-Asp-Ser amino sequence, guanidine-containing compounds, manganese(II) chloride tetrahydrate, and combinations thereof, the temperature-responsive hydrogel having a failure pressure that is at least 2 times greater than a failure pressure for a base temperature-responsive hydrogel having the same composition without the adhesion-enhancing additive.

2. The temperature-responsive hydrogel of claim 1 wherein the second monomer is described by formula 2: 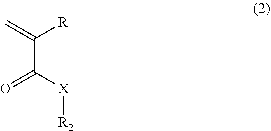
R is H or C₁₋₆ alkyl; and
R₂ is H, C₁₋₆ alkyl, C₆₋₁₈ aryl, or C₄₋₁₈ heteroaryl.

3. The temperature-responsive hydrogel of claim 2 wherein R₁ and R₂ are each independently methyl, ethyl, n-propyl, iso-propyl, n-butyl, sec-butyl, or tert-butyl.

4. The temperature-responsive hydrogel of claim 2 wherein the temperature-responsive hydrogel having a failure pressure that is 2 to 6 times greater than a failure pressure for a base temperature-responsive hydrogel having the same composition without the adhesion-enhancing additive.

5. The temperature-responsive hydrogel of claim 1 wherein the adhesion-enhancing additive is Arg-Gly-Asp-Ser amino sequence.

6. The temperature-responsive hydrogel of claim 1 wherein the guanidine-containing compounds is selected from the group consisting of aganodine, agmatidine, agmatine, ambazone, amiloride, apraclonidine, aptiganel, argatroban, arginine, argininosuccinic acid, asymmetric dimethylarginine, benexate, benzamil, bethanidine, BIT225, blasticidin s, brostallicin, camostat, cariporide, chlorophenylbiguanide, cimetidine, ciraparantag, creatine, creatine ethyl ester, creatine methyl ester, creatinine, creatinolfosfate, 2-cyanoguanidine, cycloguanil, debrisoquine, dihydrostreptomycin, ditolylguanidine, E-64, ebrotidine, epinastine, eptifibatide, famotidine, glycocyamine, guanabenz, guanadrel, guanazodine, guanethidine, guanfacine, guanidine, guanidine nitrate, guanidinium chloride, guanidinium thiocyanate, 5′-guanidinonaltrindole, 6′-guanidinonaltrindole, guanidinopropionic acid, guanochlor, guanoxabenz, guanoxan, gusperimus, impromidine, kopexil, laninamivir, leonurine, lombricine, lugduname, metformin, methylarginine, mitoguazone, octopine, OUP-16, pentosidine, peramivir, phosphocreatine, picloxydine, pimagedine, polyhexamethylene guanidine, n-propyl-l-arginine, rimeporide, robenidine, saxitoxin, siguazodan, streptomycin, sucrononic acid, sulfaguanidine, synthalin, TAN-1057 A, TAN-1057 C, tegaserod, terbogrel, 1,1,3,3-tetramethylguanidine, tetrodotoxin, tomopenem, triazabicyclodecene, UR-AK49, vargulin, VUF-8430, zanamivir, and combinations thereof.

7. The temperature-responsive hydrogel of claim 1 wherein the adhesion-enhancing additive is 3-guanidinopropionic acid.

8. The temperature-responsive hydrogel of claim 1 wherein the adhesion-enhancing additive is manganese(II)) chloride tetrahydrate.

9. The temperature-responsive hydrogel of claim 1 wherein a weight percent ratio of N-aklyacrylamide to the second monomer is from about 99:1 to about 50:50.

10. The temperature-responsive hydrogel of claim 1 wherein the poly(N-alkyacrylamide) copolymer, has a number average molecular weight of about 5,000 to about 5,000,000 Daltons.

11. The temperature-responsive hydrogel of claim 1, wherein the poly(N-alkyacrylamide) copolymer has a number average molecular weight of about 10,000 to about 3,000,000 Daltons.

12. The temperature-responsive hydrogel of claim 1 wherein the poly(N-alkyacrylamide) copolymer is present in an amount of about 0.5 weight percent to about 50 weight percent of the total weight of the temperature-responsive hydrogel.

13. The temperature-responsive hydrogel of claim 1 wherein the poly(N-alkyacrylamide) copolymer is present in an amount of about 10 weight percent to about 60 weight percent of the total weight of the temperature-responsive hydrogel.

14. The temperature-responsive hydrogel of claim 1 wherein the adhesion-enhancing additive is present in an amount of about 0.01 weight percent to about 25 weight percent of the total weight of the temperature-responsive hydrogel.

15. The temperature-responsive hydrogel of claim 1 wherein the poly(N-isopropylacrylamide) copolymer is a block copolymer.

16. The temperature-responsive hydrogel of claim 1 wherein the poly(N-isopropylacrylamide) copolymer is a statistical or random copolymer.

17. The temperature-responsive hydrogel of claim 1 further comprising a bioactive agent.

18. The temperature-responsive hydrogel of claim 1 further comprising one or more additional monomers having formula 3 that are different than the first monomer and second monomer: 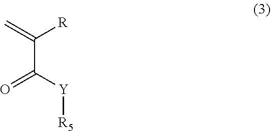
where:
Y is O or NR₆;
R is H or C₁₋₆ alkyl;
R₅ is —(CH₂)ₘ—R₇;
R₆ is H or C₁₋₆ alkyl;
R₇ is halo, hydroxyl, C₆₋₁₂ aryl, C₄₋₁₈ heteroaryl, amino, phosphorylcholinyl, or pyridinyl; and
m is an integer from 0 to 18.

19. An adhesive patch comprising the temperature-responsive hydrogel of claim 1.

20. The adhesive patch of claim 19 wherein the temperature-responsive hydrogel is deposited on a polymeric substrate.

21. The adhesive patch of claim 20 wherein the polymeric substrate is selected from the group consisting of parylene, poly-lactic acid, polyimide, and polydimethylsiloxane.

22. A method for reversibly sealing tissue damage, the method comprising:
applying a temperature-responsive hydrogel to a tear or perforation in a tissue of a subject in an amount effective to seal the tear, wherein when exposed to a temperature above its critical solution temperature, the temperature-responsive hydrogel becomes adhesive, and when exposed to a temperature below its critical solution temperature, the temperature-responsive hydrogel becomes less adhesive wherein the temperature-responsive hydrogel comprises:
water;
a poly(N-alkylacrylamide) copolymer of a first monomer having formula 1 and a second monomer that is different than the first monomer: 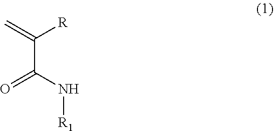
wherein:
R is H or C₁₋₆ alkyl;
R₁ is —(CH₂)ₙ—R₃, C₁₋₁₈ alkyl, C₆₋₁₈ aryl, or C₄₋₁₈ heteroaryl;
R₃ is H, hydroxyl, F, Cl, Br, NH₂, or N(R₄)₂;
R₄ is H or C₁₋₆ alkyl; n is an integer from 0 to 6 (i.e., 0, 1, 2, 3, 4, 5 or 6) and
X is O or NH; and
an adhesion-enhancing additive selected from the group consisting of Arg-Gly-Asp-Ser amino sequence, guanidine-containing compounds, manganese(II) chloride tetrahydrate, and combinations thereof, the temperature-responsive hydrogel having a failure pressure that is at least 2 times greater than a failure pressure for a base temperature-responsive hydrogel having the same composition without the adhesion-enhancing additive.

23. The method of claim 22 wherein the second monomer is described by formula 2: 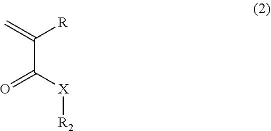
R is H or C₁₋₆ alkyl; and
R₂ is H, C₁₋₆ alkyl, C₆₋₁₈ aryl, or C₄₋₁₈ heteroaryl.

24. The method of claim 22 wherein the tissue is ocular tissue, skin, or mucosal tissue.

25. A method for reversibly sealing an ocular perforation, the method comprising:
applying a priming layer to a tear in ocular tissue of a subject, the priming layer including residues of a polyphenol, the priming layer being applied from a polyphenol solution that includes the polyphenol; and
applying a sealing layer over the priming layer the priming layer to seal the tear, the sealing layer being applied from a polymer solution that includes temperature-responsive polymer.

26. The method of claim 25 wherein the polyphenol is selected from the groups consisting of tannic acid, polydopamine, epigallocatechin gallate, epicatechin gallate, epigallocatechin, ellagic acid and trigalloylglucose.

27. The method of claim 25 wherein the sealing layer includes a component selected from the group consisting of gelatin, agarose, gellan gum, xyloglucan, k-carrageenan and synthetic polymer with UCST-type behaviors.

28. The method of claim 25 wherein the sealing layer is a temperature-responsive hydrogel that includes a poly(N-alkylacrylamide) copolymer of a first monomer having formula 1 and a second monomer that is different than the first monomer: 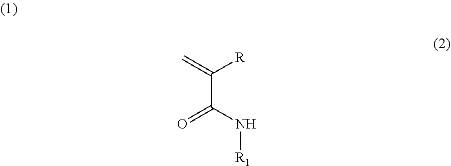
wherein:
R is H or C₁₋₆ alkyl;
R₁ is —(CH₂)ₙ—R₃, C₁₋₆ alkyl, C₆₋₁₈ aryl, or C₄₋₁₈ heteroaryl;
R₃ is H, hydroxyl, F, Cl, Br, NH₂, or N(R₄)₂;
R₄ is H or C₁₋₆ alkyl; n is an integer from 0 to 6 (i.e., 0, 1, 2, 3, 4, 5 or 6) and X is O or NH.

29. The method of claim 28 wherein the second monomer is described by formula 2: 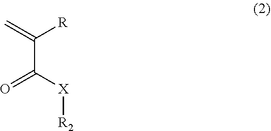
R is H or C₁₋₆ alkyl; and
R₂ is H, C₁₋₆ alkyl, C₆₋₁₈ aryl, or C₄₋₁₈ heteroaryl.

30. The method of claim 28 wherein the temperature-responsive hydrogel further comprises an adhesion-enhancing additive selected from the group consisting of Arg-Gly-Asp-Ser amino sequence (RGDS), 3-guanidinopropionic acid (GPA), manganese(II) chloride tetrahydrate, and combinations thereof, the temperature-responsive hydrogel having a failure pressure that is at least 2 times greater than a failure pressure for a base temperature-responsive hydrogel having the same composition without the adhesion-enhancing additive.

31. The method of claim 25 wherein the sealing layer includes a photothermal agent that allows release of the sealing layer by application of light.

32. The method of claim 25 wherein the tissue is ocular tissue, skin, or mucosal tissue.
